SEQUENCE LISTING (16 sequences)

SEQ ID NO: 1 - DNA, 2486 bp, Human
  CDS (124)..(1437)
  misc_feature (322)..(447): cyclin A/CDK2 binding domain
ccgggactttgcaggcagcggcggccgggggcggagcgggatcgagccctcgccgaggcctgccgccatgggcccgcgccgccgccgccgcctgtcacccgggccgcgcgggccgtgagcgtcatggccttggccggggcccctgcgggcggcccatgcgcgccggcgctggaggccctgctcggggccggcgcgctgcggctgctcgactcctcgcagatcgtcatcatctccgccgcgcaggacgccagcgccccgccggctcccaccggccccgcggcgcccgccgccggcccctgcgaccctgacctgctgctcttcgccacaccgcaggcgccccggcccacacccagtgcgccgcggcccgcgctcggccgcccgccggtgaagcggaggctggacctggaaactgaccatcagtacctggccgagagcagtgggccagctcggggcagaggccgccatccaggaaaaggtgtgaaatccccgggggagaagtcacgctatgagacctcactgaatctgaccaccaagcgcttcctggagctgctgagccactcggctgacggtgtcgtcgacctgaactgggctgccgaggtgctgaaggtgcagaagcggcgcatctatgacatcaccaacgtccttgagggcatccagctcattgccaagaagtccaagaaccacatccagtggctgggcagccacaccacagtgggcgtcggcggacggcttgaggggttgacccaggacctccgacagctgcaggagagcgagcagcagctggaccacctgatgaatatctgtactacgcagctgcgcctgctctccgaggacactgacagccagcgcctggcctacgtgacgtgtcaggaccttcgtagcattgcagaccctgcagagcagatggttatggtgatcaaagcccctcctgagacccagctccaagccgtggactcttcggagaactttcagatctcccttaagagcaaacaaggcccgatcgatgttttcctgtgccctgaggagaccgtaggtgggatcagccctgggaagaccccatcccaggaggtcacttctgaggaggagaacagggccactgactctgccaccatagtgtcaccaccaccatcatctcccccctcatccctcaccacagatcccagccagtctctactcagcctggagcaagaaccgctgttgtcccggatgggcagcctgcgggctcccgtggacgaggaccgcctgtccccgctggtggcggccgactcgctcctggagcatgtgcgggaggacttctccggcctcctccctgaggagttcatcagcctttccccaccccacgaggccctcgactaccacttcggcctcgaggagggcgagggcatcagagacctcttcgactgtgactttggggacctcacccccctggatttctgacagggcttggagggaccagggtttccagagtagctcaccttgtctctgcagccctggagccccctgtccctggccgtcctcccagcctgtttggaaacatttaatttatacccctctcctctgtctccagaagcttctagctctggggtctggctaccgctaggaggctgagcaagccaggaagggaaggagtctgtgtggtgtgtatgtgcatgcagcctacacccacacgtgtgtaccgggggtgaatgtgtgtgagcatgtgtgtgtgcatgtaccggggaatgaaggtgaacatacacctctgtgtgtgcactgcagacacgccccagtgtgtccacatgtgtgtgcatgagtccatctctgcgcgtgggggggctctaactgcactttcggcccttttgctcgtggggtcccacaaggcccagggcagtgcctgctcccagaatctggtgctctgaccaggccaggtggggaggctttggctggctgggcgtgtaggacggtgagagcacttctgtcttaaaggttttttctgattgaagctttaatggagcgttatttatttatcgaggcctctttggtgagcctggggaatcagcaaaaggggaggaggggtgtggggttgataccccaactccctctacccttgagcaagggcaggggtccctgagctgttcttctgccccatactgaaggaactgaggcctgggtgatttatttattgggaaagtgagggagggagacagactgactgacagccatgggtggtcagatggtggggtgggccctctccagggggccagttcagggcccagctgccccccaggatggatatgagatgggagaggtgagtgggggaccttcactgatgtgggcaggaggggtggtgaaggcctcccccagcccagaccctgtggtccctcctgcagtgtctgaagcgcctgcctccccactgctctgccccaccctccaatctgcactttgatttgcttcctaacagctctgttccctcctgctttggttttaataaatattttgatgacgtt

SEQ ID NO: 2 - protein, 437 aa, Human
MALAGAPAGGPCAPALEALLGAGALRLLDSSQIVIISAAQDASAPPAPTGPAAPAAGPCDPDLLLFATPQAPRPTPSAPRPALGRPPVKRRLDLETDHQYLAESSGPARGRGRHPGKGVKSPGEKSRYETSLNLTTKRFLELLSHSADGVVDLNWAAEVLKVQKRRIYDITNVLEGIQLIAKKSKNHIQWLGSHTTVGVGGRLEGLTQDLRQLQESEQQLDHLMNICTTQLRLLSEDTDSQRLAYVTCQDLRSIADPAEQMVMVIKAPPETQLQAVDSSENFQISLKSKQGPIDVFLCPEETVGGISPGKTPSQEVTSEEENRATDSATIVSPPPSSPPSSLTTDPSQSLLSLEQEPLLSRMGSLRAPVDEDRLSPLVAADSLLEHVREDFSGLLPEEFISLSPPHEALDYHFGLEEGEGIRDLFDCDFGDLTPLDF

SEQ ID NO: 3 - DNA, 1766 bp, Human
  CDS (429)..(1742)
  misc_feature (621)..(743): cyclin A/CDK2 binding domain
caggactagagagcgagccgcaaggaagtcggtgcagtcgagacccccctccccatcccagcgcatcgcgtctccgccgagcttgagggcacgccggggacccctccccagagccggccggaccccaggtgccgaggccttggggagcgcggggcgtcccgggtcgcggtgccctcgggacgagacagcccctggcagtgccaccaccgcagccgccgggcgatctccaagcggcgatctctaagcgctgctctctgctcggccgcgggccaggaggggagggtccggccttgccccgcaggcgtccattggcggcttcccccggcctccgcgccatgccgcgggccgtgtgaaaggcggcagcaccggaacccgcaggtgtccgcgggcgcgccaagcccttttgggtagggggcgccttactcgctatgctgcaagggccccgggccttggcttcggccgctgggcagaccccgaaggtggtgcccgcgatgagccccacagagctgtggccatccggcctcagcagcccccagctctgcccagctactgctacctactacacaccgctgtacccgcagacggcgcctcccgcagcggcgccaggcacctgcctcgacgccactccccacggacccgagggccaagttgtgcgatgcctgccggcaggccggctgccggccaaaaggaagctggatctggaggggattgggaggcccgtcgtccctgagttcccaacccccaaggggaagtgcatcagagtggatggcctccccagccccaaaacccccaaatcccccggggagaagactcggtatgacacttcgctggggctgctcaccaagaagttcatttacctcctgagcgagtcagaggatggggtcctggacctgaactgggccgctgaggtgctggacgtgcagaagcggcgcatctatgacatcaccaacgtgctggaaggcatccagctcatccgcaagaaggccaagaacaacatccagtgggtaggcaggggaatgtttgaagaccccaccagacctgggaagcagcaacagctggggcaggagctgaaggagctgatgaacacggagcaggccttggaccagctcatccagagctgctctctgagcttcaagcacctgactgaggacaaggccaacaagaggctggcctatgtgacttaccaggatatccgtgctgttggcaactttaaggagcagacagtgattgccgtcaaggcccctccgcagacgagactggaagtgcccgacaggactgaggacaacctgcagatatatctcaagagcacccaagggcccatcgaagtctacctgtgcccagaggaggtgcaggagccggacagtccttccgaggagcctctcccctctacctccaccctctgccccagccctgactctgcccagcccagcagcagcaccgaccctagcatcatggagcccacagcatcctcagtgccagcaccagcgccaaccccccagcaggccccaccgcctccatccctggtccccttggaggctactgacagcctgctggagctgccgcacccactcctgcagcagactgaggaccagttcctgtccccgaccctggcgtgcagctcccctctgatcagcttctccccatccttggaccaggacgactacctgtggggcttggaggcgggtgagggcatcagcgatctcttcgactcctacgaccttggggacctgttgattaattgagtggccctgcctgcccccagcagc

SEQ ID NO: 4 - protein, 437 aa, Human
MLQGPRALASAAGQTPKVVPAMSPTELWPSGLSSPQLCPATATYYTPLYPQTAPPAAAPGTCLDATPHGPEGQVVRCLPAGRLPAKRKLDLEGIGRPVVPEFPTPKGKCIRVDGLPSPKTPKSPGEKTRYDTSLGLLTKKFIYLLSESEDGVLDLNWAAEVLDVQKRRIYDITNVLEGIQLIRKKAKNNIQWVGRGMFEDPTRPGKQQQLGQELKELMNTEQALDQLIQSCSLSFKHLTEDKANKRLAYVTYQDIRAVGNFKEQTVIAVKAPPQTRLEVPDRTEDNLQIYLKSTQGPIEVYLCPEEVQEPDSPSEEPLPSTSTLCPSPDSAQPSSSTDPSIMEPTASSVPAPAPTPQQAPPPPSLVPLEATDSLLELPHPLLQQTEDQFLSPTLACSSPLISFSPSLDQDDYLWGLEAGEGISDLFDSYDLGDLLIN

SEQ ID NO: 5 - DNA, 4744 bp, Human
  CDS (67)..(1464)
  misc_feature (367)..(525): cyclin A/CDK2 binding domain
aaataataaagaaattgaaaacaatacattaatataccataacactaaaaagagcaggagcgagagatgagaaagggaatccagcccgctctggagcagtacctggtgaccgccgggggtggggagggggcggctgtcgtcgccgccgccgctgcagcctccatggacaaaagggcactgctagccagccccggcttcgccgccgccgccgccgctgccgccgccccgggcgcgtacatccagatcctcaccacgaacacttccaccacctcctgttcctcctccctccaaagcggcgccgtagccgccggccccctcctccccagtgcccccggcgcggagcagaccgccggcagcctcctctacaccacgccgcacggaccctccagcagagccgggctgctgcagcagccaccagcgctgggacgcggcggcagcggcggcggcggcggccctccggcaaagcgaaggctggagctaggagaaagcggtcatcagtacctctcagatggtttaaaaacccccaagggcaaaggaagagctgcactacgaagtccagatagtccaaaaactccaaaatctccctcagaaaaaacgcggtatgatacgtctcttggtctgctcaccaagaagttcattcagctcctgagccagtcacccgatggggtattggatttgaacaaggcagcagaagtgctaaaagtgcaaaagagaaggatttatgatatcaccaacgttctggaaggcatccacctcattaagaagaagtctaaaaacaacgtccaatggatgggctgcagtctgtctgaggatgggggcatgctggcccagtgtcaaggcctgtcaaaagaagtgaccgagctcagtcaggaagagaagaaattagatgaactgatccaaagctgcaccctggacctcaaactgttaaccgaggattcagagaatcaaaggttagcttatgttacatatcaagatattcgaaaaattagtggccttaaagaccaaactgttatagttgtgaaagcccctccagaaacaagacttgaagtgcctgactcaatagagagcctacaaatacatttggcaagtacccaagggcccattgaggtttacttatgtccagaagagactgaaacacacagtccaatgaaaacaaacaaccaagaccacaatgggaatatccctaaacccgcttccaaagacttggcttcaaccaactcaggacatagcgattgctcagtttctatgggaaacctttctcctctggcctccccagccaacctcttacagcagactgaggaccaaattccttccaacctagaaggaccgtttgtgaacttactgcctcccctgctgcaagaggactatctcctgagcctcggggaggaggaaggcatcagcgatctcttcgatgcttacgatttggaaaagctcccactggtggaagacttcatgtgtagttgattatgcttcgtgtgaactctccttaaaaaccgatatttttttatcatggaccagaacatctgtcatgcagtgttgtcccttcctaccttcttcctccaagagagtatcatgaagtaaactacaaacttcagaagaaagctgacattttaatgaattttttaaaaaattaataaacaaattgtctaaacgcacagttgcaggctcccttgggaaagccctgctttgctccaggctccaagatctcctggctaagtcagcaagtgagaaaatgtgcaatcaggtgtctctcaccccgaattgtccttcctccttcctccccggattggcttgctgtgcctgacggatgggctgtagaatggggtctggccacctggcctgctgggaaacagcaatcttccttaatagcatttcaagccgtgccttctccgcagaatgcatgtctttgaggtctgctaatatggaatggaactgcagcaaatgcaaacttgaagtcatgcaaaagtatgaaatggatttcttcagctcttcttaggaatatttaaattactgtcataattcagtttaagctatgaactgtgtgtcccagtaggaggtcaagaaaacctccacagccttctggatgaagaacctgttttcaaatatacttgttgcagatacagaagactagtagagttctgccactctaagctgttgtggattttcctgtctccatgaaccactcccattcccccgtccccaatgtgtttgtgagtttccagttgatttgtagcaaatgcctacttagttctttgtggattgttctagacttttaatttttttagctgccatttaagcattcctgtggcacccatcaccatttcaatttaattgtttactttgaagcggtttttgcaaattcatattacttaagcagagggagagaacctctactgatcagagcatctaaacctgtgtgatctaaggtttatcagcctctgcaaggagctttgtcccatcgtgcttccattcccaggagggggagcttggagcgagtcagtcctggggcttgctgacatgggtggcccattggaaaggagaaccaggtcagatgatgtaacagccccaaggagcagcaggcatgggtccctccatccttgggcttcccgggcccctgtgacaggggaaagggctctcttacaccgcactcagggagaccacttctcaggatggggtcagatggagagacctctagggagaaagacatccccattgtgtgagtggcatttccttaagctggcaggaacagggagcagccctgtgtcgggggctggaatagttctggccagaccccgttccccttcctctatgaaggaataagttggaccaagggaagtcggggacgtaaaaaatgaagcaaaacaatgccagggtgtctcccgctttactcttcaggaatggtgtcccaagttggaggctttgtgtcagctgcaaatcctaccagttatgtccaagaatggctttccctcgggcaggtggcagcggccatctcccactgggaatatggcgtagtatctccggtccattccttggatgctaaggactgcgggaatgagggagtcagataaagaacaaacctcgaaacgaacagttaaattgaaatgctatgtgcctgacccaatggtaggcacatagtaggcactcaactcatatgtttaattgaattgaaaatatcccttaggaaaaaaaaaaaacacacaaaaaaccacaagagccccagccagtttactccaggtagatttccacaatatgcaaagtggtggtggggtcaagacagatgacaccagcactttaaactctttgtgtgggtatgcgtgggtgtatgtttgggaagaaaaacaaaggtgcagactatcttcctttttttcttcttcagcctccatccctggcctcctcccctcacacacactggacttggtacaaaatgtcggtgtggtcctagatgaagcattggggtgggggagggagagggagctttgtgttaagtgcctactggaaatgcactgtggggttttttcctgtatgggaaaccatttatgccaagcttttccccatttcccatatttatctcatctggttagctgcctctgcttccagctttgtgtaattctctttgccagctgcacaaagctgattttttccaaagtctaaagactgagctcacctggctagattgttgtgtgttttgttgaattttttcataatgtaatgccgtatttattgtttttaaaatgaaaggaatactaataagtcttaaaagttccttcatgcataagatttttttccagttactgggcttaactggtgtacattaattagatgtccatactgtattttgtttgcattaagtaattttctttttgacttagtatccggcacacaaagtgggttagtactacagtatttgcgttactttaagtactaagtatgcaggtttcctggtaccattgagttgctgctattaaagctcacacacgaaatggctaaaagttacaagtgtgcaaattatgactgcgtgagccttagaaaataaaatgtataaagggcaacacatgagctgtcaaacagtgttaggagtgtgtttatatgtacagagttgtgcatagcaatcgttttatttaagttgatatgtagtctactcacattttcattatttagcaattttgtacaaaaatagcaattaatttgtaaacactgccagaatactttctagctgctttgtaattttttaagagtgttattttgtttttgtttttctgttctttgttgtggctcttgttttcatttttgttgtacgtgtagatctgtaaataaaattgcagtatttaaagcttaagctttcaggaaaaagaaaataagaattcagtgtgtgcatgacaactcgtgtgtatgagaaggagggatatgaaggaagatggcttgcagagtaagtcgggtggcaattgtcagggtgtgggaatttcttttcctacggggtacgtgattttgtaaaaaggaagtatttctcccaaaattgggagtaggcaaactactaatcagtttagctttgtgttgtatgctagtttaaaaaagaaaatatgtaatataatgtaaaaaaaaacaaaaaaaagcttttatgatggattttgtaaatagatttgttacagggtgacctgttctctagctgtgatcttaccacttcaaatgggtgtaatttgaataaattttgtatggtaaaggatcaataaaatgattttttttaaga

SEQ ID NO: 6 - protein, 465 aa, Human
MRKGIQPALEQYLVTAGGGEGAAVVAAAAAASMDKRALLASPGFAAAAAAAAAPGAYIQILTTNTSTTSCSSSLQSGAVAAGPLLPSAPGAEQTAGSLLYTTPHGPSSRAGLLQQPPALGRGGSGGGGGPPAKRRLELGESGHQYLSDGLKTPKGKGRAALRSPDSPKTPKSPSEKTRYDTSLGLLTKKFIQLLSQSPDGVLDLNKAAEVLKVQKRRIYDITNVLEGIHLIKKKSKNNVQWMGCSLSEDGGMLAQCQGLSKEVTELSQEEKKLDELIQSCTLDLKLLTEDSENQRLAYVTYQDIRKISGLKDQTVIVVKAPPETRLEVPDSIESLQIHLASTQGPIEVYLCPEETETHSPMKTNNQDHNGNIPKPASKDLASTNSGHSDCSVSMGNLSPLASPANLLQQTEDQIPSNLEGPFVNLLPPLLQEDYLLSLGEEEGISDLFDAYDLEKLPLVEDFMCS

SEQ ID NO: 7 - DNA, 1332 bp, Human
  CDS (63)..(1304)
  misc_feature (108)..(800): Transcription factor E2F/dimerisation partner (TDP)
gcgcggaagtggcgcggcgcgcctggcctggcctggctgaggggaggcggcgggcgggcgcgatggcggaggccgggccacaggcgccgccgcccccgggcactccaagccggcacgaaaagagcctgggactgctcaccaccaagttcgtgtcccttctgcaggaggccaaggacggcgtgcttgacctcaagctggcagctgacaccctagctgtacgccagaagcggcggatttacgacattaccaatgttttggaaggtatcgggctaatcgagaaaaagtccaagaacagcatccagtggaagggtgtggggcctggctgcaatacccgggagattgctgacaaactgattgagctcaaggcagagatcgaggagctgcagcagcgggagcaagaactagaccagcacaaggtgtgggtgcagcagagcatccggaacgtcacagaggacgtgcagaacagctgtttggcctacgtcactcatgaggacatctgcagatgctttgctggagataccctcttggccatccgggccccatcaggcaccagcctggaggtgcccatcccagagggtctcaatgggcagaagaagtaccagattcacctgaagagtgtgagtggtcccattgaggttctgctggtgaacaaggaggcatggagctcaccccctgtggctgtgcctgtgccaccacctgaagatttgctccagagcccatctgctgtttctacacctccacctctgcccaagcctgccctagcccagtcccaggaagcctcacgtccaaatagtcctcagctcactcccactgctgtccctggcagtgcagaagtccagggaatggctggcccagcagctgagatcacagtgagtggcggccctgggactgatagcaaggacagtggtgagctcagttcactcccactgggcccaacaacactggacacccggccactgcagtcttctgccctgctggacagcagcagcagcagcagcagcagcagcagcagcagcagcaacagtaacagcagcagttcgtccggacccaacccttctacctcctttgagcccatcaaggcagaccccacaggtgttttggaactccccaaagagctgtcagaaatctttgatcccacacgagagtgcatgagctcggagctgctggaggagttgatgtcctcagaagtgtttgcccctctgcttcgtctttctccacccccgggagaccacgattatatctacaacctggacgagagtgaaggtgtctgtgacctctttgatgtgcctgttctcaacctctgactgacagggacatgccctgtgtggctgg

SEQ ID NO: 8 - protein, 413 aa, Human
MAEAGPQAPPPPGTPSRHEKSLGLLTTKFVSLLQEAKDGVLDLKLAADTLAVRQKRRIYDITNVLEGIGLIEKKSKNSIQWKGVGPGCNTREIADKLIELKAEIEELQQREQELDQHKVWVQQSIRNVTEDVQNSCLAYVTHEDICRCFAGDTLLAIRAPSGTSLEVPIPEGLNGQKKYQIHLKSVSGPIEVLLVNKEAWSSPPVAVPVPPPEDLLQSPSAVSTPPPLPKPALAQSQEASRPNSPQLTPTAVPGSAEVQGMAGPAAEITVSGGPGTDSKDSGELSSLPLGPTTLDTRPLQSSALLDSSSSSSSSSSSSSNSNSSSSSGPNPSTSFEPIKADPTGVLELPKELSEIFDPTRECMSSELLEELMSSEVFAPLLRLSPPPGDHDYIYNLDESEGVCDLFDVPVLNL

SEQ ID NO: 9 - DNA, 1752 bp, Human
  CDS (35)..(1075)
  misc_feature (173)..(388): DNA binding domain
cgggggggcccgaccaccgcggggccgggacgcgatggcggcggcagagcccgcgagctcgggccagcaggcgccggcagggcaggggcagggccagcggccgccgccgcagcctccgcaggcgcaagccccgcagccgcccccgccgccgcagctcgggggcgcggggggcggcagcagcaggcacgagaagagcctggggctgctcactaccaagttcgtgtcgctgctgcaggaggccaaggacggcgttctggatctcaaagcggctgctgatactttggctgtgaggcaaaaaaggagaatttatgatatcaccaatgtcttagagggaattgacttgattgaaaaaaagtcaaaaaacagtatccagtggaaaggtgtaggtgctggctgtaatactaaagaagtcatagatagattaagatatcttaaagctgaaattgaagatctagaactgaaggaaagagaacttgatcagcagaagttgttgctacagcaaagcatcaaaaatgtgatggacgattccattaataatagattttcctatgtaactcatgaagacatctgtaattgctttaatggtgatacacttttggccattcaggcaccttctggtacacaactggaggtacccattccagaaatgggtcagaatggacaaaagaaataccagatcaatctaaagagtcattcaggacctatccatgtgctgcttataaataaagagtcgagttcatctaagcccgtggtttttcctgttcccccacctgatgacctcacacagccttcctcccagtccttgactccagtgactccacagaaatccagcatggcaactcaaaatctgcctgagcaacatgtctctgaaagaagccaggctctgcagcagacatcagctacagatatatcttcagcaggatctattagtggagatatcattgatgagttaatgtcttctgacgtgtttcctctcttaaggctttctcctaccccggcagatgactacaactttaatttagatgataacgaaggagtttgtgatctgtttgatgtccagatactaaattattagattccatggaaacttgggactgttatctacctctaactgtgtaacattttagacttcttaataacctaaatatttaaaataatgaatgtaacaccttttttagttcactgattctgaagtgttcttccctaatactttctttacttcacaaaacttcaaccataaaaacaaagggctctgattgctttaggggataagtgatttaatattcacaaacgtccccactcccaaaagtaactatattctggatttcaacttttcttctaattgtgaatccttccgttttttcttcttaaggaggaaagttaaaggacactacaggtcatcaaaaacaagttggccaaggactcattacttgtcttatatttttactgccactaaactgcctgtatttctgtatgtccttctatccaaacagacgttcactgccacttgtaaagtgaaggatgtaaacgaggatatataactgtttcagtgaacagattttgtgaagtgccttctgttttagcactttaagtttatcacattttgttgacttctgacattccactttcctaggttataggaaagatctgtttatgtagtttgtttttaaaatgtgccaatgcctgtacattaacagatttttaaaaataaaattgtataaaacattaaaaaaaaaaaaaaaaaaaaaaaaa

SEQ ID NO: 10 - protein, 346 aa, Human
MAAAEPASSGQQAPAGQGQGQRPPPQPPQAQAPQPPPPPQLGGAGGGSSRHEKSLGLLTTKFVSLLQEAKDGVLDLKAAADTLAVRQKRRIYDITNVLEGIDLIEKKSKNSIQWKGVGAGCNTKEVIDRLRYLKAEIEDLELKERELDQQKLLLQQSIKNVMDDSINNRFSYVTHEDICNCFNGDTLLAIQAPSGTQLEVPIPEMGQNGQKKYQINLKSHSGPIHVLLINKESSSSKPVVFPVPPPDDLTQPSSQSLTPVTPQKSSMATQNLPEQHVSERSQALQQTSATDISSAGSISGDIIDELMSSDVFPLLRLSPTPADDYNFNLDDNEGVCDLFDVQILNY

SEQ ID NO: 11 - DNA, 2043 bp, Human
  CDS (1)..(846)
  misc_feature (148)..(387): DNA binding domain
atgagtcagcagcggccggcgaggaagttacccagtctcctcctggacccgacggaggagacggttcgccgtcggtgccgagaccccatcaacgtggagggcctgctgccatcaaaaataaggattaatttagaagataatgtacaatatgtgtccatgagaaaagctctaaaagtgaagagacctcgttttgatgtatcgctggtttatttaactcgaaaatttatggatcttgtcagatctgctcccgggggtattcttgacttaaacaaggttgcaacgaaactgggagtccgaaagcggagagtgtatgacatcaccaatgtcttagatggaatcgacctcgttgaaaagaaatccaagaaccatattagatggataggatctgatcttagcaattttggagcagttccccaacaaaagaagctacaggaggaactttctgacttatcagcaatggaagatgctttggatgagttaattaaggattgtgctcagcagctgtttgagttaacagatgacaaagaaaatgaaagactagcatatgtgacctatcaagacattcatagcattcaggccttccatgaacagatcgtcattgcagttaaagctccagcagaaaccagattggatgttccagctcccagagaagactctatcacagtgcacataaggagcaccaacggacctatcgatgtctatttgtgtgaagtggagcagggtcagaccagtaacaaaaggtctgaaggtgtcgggacctcttcatctgagagcactcatccagaaggccctgaggaagaagaaaatcctcagcaaagtgaagaattgcttgaagtaagcaactgatggcatttgagaatttatgtatcactgagttttttgggaatatcttcgtggagaattacgcatcaaatttgattctcagagcaataaattatccatgaagtgctctcgttctcagtagcggcatcatggccagtagtgtctttgaggagttcaccacttagattactgagtaattgtggtttccacatttgaaaacaactccttttataattattcactgctttttgtcagtgaaatagacatcttgcctcctcaagtagcttcatcacagagtgtcatgaagacagacagtcaggctgaaatggacagttctttgtggactctacccttcccttcaaggagtatgtcatatatcacaaaagaaattgccttacactggttcacgtttgcagttacctgttgtacattgcatagatgtacacacgaatttaaatgtgatgtctttgtatatatctgtataatgttgagattacttacgaaatatgtctgagtgacacttttcacccttgtacagccaaaataatgtatatatggaaagtgacagacaaattctctaatctctttggtatctataacttattagaatcctctggatgagggttagaagagactttttccaaacttctacatgtagaagtatcataaatgtgctacacatttatgtttgtggatttaattaaagtattttaatatggttttcagtgctaaaattggagtcagatacttcttggttttaagctgtctacctaattgctgtctcccagcagactggtggcatgcccagtggctttgggggcaaggatagaaatgccatcaggaaatagctgaattcattgtgaaacatgaattcagtcatggtgataattggaaactcctttcaggtttttgcaagtagattttgtaatgtttgtgtatgcagccttgctgttgagtcagtccaaggggttttacttaggacaagttgtaccttgccctctctccagctctgctcccacattttcacatacctagctgtttctacctcattgggtaactcatttaccactctgtgcctcagtttactctgtagtttaccattagactgtgagctccttgagggactttgtcataatcactgttacatcccagtgcctcacaccatgcctggcccttaagaagtgctcaataaatgtctgaacaaataaaaaaaaaaaaaaa

SEQ ID NO: 12 - protein, 281 aa, Human
MSQQRPARKLPSLLLDPTEETVRRRCRDPINVEGLLPSKIRINLEDNVQYVSMRKALKVKRPRFDVSLVYLTRKFMDLVRSAPGGILDLNKVATKLGVRKRRVYDITNVLDGIDLVEKKSKNHIRWIGSDLSNFGAVPQQKKLQEELSDLSAMEDALDELIKDCAQQLFELTDDKENERLAYVTYQDIHSIQAFHEQIVIAVKAPAETRLDVPAPREDSITVHIRSTNGPIDVYLCEVEQGQTSNKRSEGVGTSSSESTHPEGPEEEENPQQSEELLEVSN

SEQ ID NO: 13 - DNA, 3624 bp, Mouse
  CDS (226)..(2940)
  misc_feature (649)..(1137): Transcription factor E2F/dimerisation partner (TDP) 1
gttttagcggggaccgcgatccagggctggagcttggcttaggcgggtaggagacgctgtcgctacgcggacaccgcgggtggagcggaggatctgcgagccttgccctgccctgcccatgtcgccagccggccgctgagatctacacgctctgcccgctcacgcgtgcggctgcggacgtccccggagagaccctccatcgcgcagcgacattgtgtgttccagatggaggtgaattgtttaacactaaaagacttgatcagccccaggcagaccagactagattttgccattgaagatgcagagaacgcacaaaaggaaaatatatttgtcgaccgatcaaggatgaccccaaagacaccgatgaagaacgagccgatcgacctgtcaaagcaaagaatcttcaccccagacagaaaccccattactccagtaaagccggtcgacaggcagccgcaggtggagccctggacacccacagccaacctgaagatgctcatcagcgccgccagcccagacataagagaccgggagaagaaaaaggagctgttcagacccattgagaataaggaggatgcgttcgtgaactccctgcagcttgacgtggccggtgacggcgctgtggatgagtatgagaagcaaaggccaagcaggaaacagaagagcttggggctgctgtgccagaagtttctagctcgctatccgagctaccccttgtcaacggagaaaaccaccatctccctagatgaggtcgccgtcagtctcggcgtggaaaggagacgcatctatgacatcgtaaacgtgctggagtcgctgcatctggtcagccgggtggctaagaatcagtatggttggcacggtcggcacagcctccccaaaaccctgaggaccctacagagactgggagaggaacagaaatacgaggagcagatggcctgcctccagcagaaggagctggacctgatggggtataggttcggagaacgcaggaaagacgggtctccggacccccgagatccacacctacttgatttttccgaagccgactacccctcttcatctgcaaacagtcgaaaagataaatctttaagaattatgagccagaagtttgtcatgctgttcctcgtctccaaaaccaagattgtcaccctggatgtagctgccaaaatcctcatagaagaaagccaagataccccggaccacagcaaattcaaaacaaaggtacggaggctctatgacatagccaatgttctgaccagcctggctctgataaagaaagttcacgtaacagaagagcgaggccggaaaccagccttcaagtggatcgggcctgtggacttcagctccattgatgaagaactgttggacgtctctgcatccatcttaccagaattgaagaaggaagcatacggccagatccgagtgtgtgcaaaagagaggctggtgcggtacggttcctttaacacggttcacacgtctgagaagatacagagaaaagtgagctcagaacccagcagcccacagggagaaagacaagggtcagcctattccctggaaattggaagtctggcggccatctacagacagaaagtagaagataattcgcaggaggaagcctttgtcagtaacacagcagtgcctccagcaagcatcttggaccccgccctctccatggactctgaatactgtgtcaagcctttagcccagccagtattttctgttgctcaaacagatttgccggccttctctgcgcagaatggtccaagcggacaagtaggtgtccccgttccttctgcagcctccgacacagagaacttaaagccagctctgctcgccggccagcccctggtctacgtgccctccacccaactcttcatgctgtacgggagtgtgcaggagggactgtccccagagtcaaggtcagaggaagatggtggtggctccgatgtcccagctgacctgtcagtgacaccctctgctcagaagcgcctgtgtgaggagagagatcctcaggaggaggaggacgagcccgccatgaaaagacaaagtcaggaatttgaagacagccccttatccctagttatgcccaagaaaccctcaagttccacagaccttgcctgtcctgtgaccatgggaaacgggagctctccacccctggaagacgcctgtgttaagggtcagctccctgctgcagaagaagttacaggaaaggctgccccaaactgctatgttgcttctgagtgtggaaacccggcaagaaatccagacactgaaaagccttccaacgaaaacgagatcaccaaagacccctcgctgatgcagtacctgtatgtgcagtctcccgcagggttaaatggttttaacatggttttaccaggcacccagaccccccacaccgtggcaccctccccggctcagctgccatccttcggcgtaccctgcatgttcctgcaatccccaggcctgggcccttttccagttctctattctcccgcaatacccgggcccatttcttcagcccacggcactcacccaaaccccggacccatgaacttcggcttgtccaccctggcatctgcttctcaccttctcatcagcccggcagccatggtcaatccaaagccatccaccctcccctgtacagaccctcagcttcgctgtcagccctcactcaacctgaaccccgtgatgccaggctcacacggcgtcatccaccccgagtccccctgttacgtgagacatccggtatcgatggtaaaggcagagcagtcccctgcaccggcgactcccaagagcatccaacggagacatcgggagacgttcttcaagactcccggcagcctcggcgatccggtctttagaagaaaagaaaggaaccaatcaagaaacactagctcagcccagaggagactagaaatctctagcagcgggcctgactagcctgatgcttcggtgggtgggatggcctcagtgcctgacgctccctgtgtcccagacaaatgtcctaagtggaacacaggtcaccccaggtcctcagctgtgtccttgctctcttgctcgatccactggttctgctaacttcccgatatcatgagtcatctatcttcctggaagtcattaatactaattatgcatttaacaccacctagcatccaggctgtgcccgtggcggtgttgctgcggcaatgccgtgtgactgctatggtttttctgaagcggggttgcctagcacttaaccagggtaagtgtcggcgatatgcctctccacctgccctgagcaagtctgtagtgagaactgtatctgcagcctaaatacgctactgcttcctaaggctccgggaggccaccttcagtgaggaatcctatggtattatttatttttgtccctgaagcgggactttactgcacaaagtttacaacatcatccaatgtatttttcagggtattgcgacttgaatgtttatgcttttgttgttgttgttattatgacttgccctgcacttattctacagcttattaataatgtggataaatgtatatacacgataaaagtcaagaccaaaataactgtgaatgctacacctagccattaataaactgtgcagacccctctggagttgcagg

SEQ ID NO: 14 - protein, 904 aa, Mouse
MEVNCLTLKDLISPRQTRLDFAIEDAENAQKENIFVDRSRMTPKTPMKNEPIDLSKQRIFTPDRNPITPVKPVDRQPQVEPWTPTANLKMLISAASPDIRDREKKKELFRPIENKEDAFVNSLQLDVAGDGAVDEYEKQRPSRKQKSLGLLCQKFLARYPSYPLSTEKTTISLDEVAVSLGVERRRIYDIVNVLESLHLVSRVAKNQYGWHGRHSLPKTLRTLQRLGEEQKYEEQMACLQQKELDLMGYRFGERRKDGSPDPRDPHLLDFSEADYPSSSANSRKDKSLRIMSQKFVMLFLVSKTKIVTLDVAAKILIEESQDTPDHSKFKTKVRRLYDIANVLTSLALIKKVHVTEERGRKPAFKWIGPVDFSSIDEELLDVSASILPELKKEAYGQIRVCAKERLVRYGSFNTVHTSEKIQRKVSSEPSSPQGERQGSAYSLEIGSLAAIYRQKVEDNSQEEAFVSNTAVPPASILDPALSMDSEYCVKPLAQPVFSVAQTDLPAFSAQNGPSGQVGVPVPSAASDTENLKPALLAGQPLVYVPSTQLFMLYGSVQEGLSPESRSEEDGGGSDVPADLSVTPSAQKRLCEERDPQEEEDEPAMKRQSQEFEDSPLSLVMPKKPSSSTDLACPVTMGNGSSPPLEDACVKGQLPAAEEVTGKAAPNCYVASECGNPARNPDTEKPSNENEITKDPSLMQYLYVQSPAGLNGFNMVLPGTQTPHTVAPSPAQLPSFGVPCMFLQSPGLGPFPVLYSPAIPGPISSAHGTHPNPGPMNFGLSTLASASHLLISPAAMVNPKPSTLPCTDPQLRCQPSLNLNPVMPGSHGVIHPESPCYVRHPVSMVKAEQSPAPATPKSIQRRHRETFFKTPGSLGDPVFRRKERNQSRNTSSAQRRLEISSSGPD

SEQ ID NO: 15 - DNA, 366 bp, Human
  CDS (1)..(363)
atgggaaaaggtgtgaaatccccgggggagaagtcacgctatgagacctcactgaatctgaccaccaagcgcttcctggagctgctgagccactcggctgacggtgtcgtcgacctgaactgggctgccgaggtgctgaaggtgcagaagcggcgcatctatgacatcaccaacgtccttgagggcatccagctcattgccaagaagtccaagaaccacatccagtggctgggcagccacaccacagtgggcgtcggcggacggcttgaggggttgacccaggacctccgacagctgcaggagagcgagcagcagctggaccacctgatgaatatctgtactacgcagctgcgcctgctctcctga

SEQ ID NO: 16 - protein, 121 aa, Human
MGKGVKSPGEKSRYETSLNLTTKRFLELLSHSADGVVDLNWAAEVLKVQKRRIYDITNVLEGIQLIAKKSKNHIQWLGSHTTVGVGGRLEGLTQDLRQLQESEQQLDHLMNICTTQLRLLS

(19) United States
(12) Patent Application Publication
(10) Pub. No.: US 2005/0245473 A1
(43) Pub. Date: Nov. 3, 2005

(54) Proliferation- and differentiation-modulating agents and uses therefor

(76) Inventor: Nicholas Andrew Saunders, Holland Park (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/967,648

(22) Filed: Oct. 15, 2004

Related U.S. Application Data
(60) Provisional application No. 60/512,010, filed on Oct. 16, 2003.

(51) Int. Cl. A61K 48/00; C12N 15/85

(52) U.S. Cl. 514/44; 435/455

(57) ABSTRACT

The present invention discloses the use of E2F pathway modulators and optionally a differentiation stimulus in methods for treating or preventing conditions associated with the deregulation of epithelial cell proliferation and differentiation and for diagnosing the presence or risk of developing such conditions.

### RELATED APPLICATION

This application is a non-provisional application claiming priority under 35 U.S.C. §119(e) from Provisional Application No. 60/512,010, filed Oct. 16, 2003.

### FIELD OF THE INVENTION

THIS INVENTION relates generally to cell proliferation and differentiation. More particularly, the present invention relates to conditions associated with the deregulation of cellular, especially epithelial, and more especially keratinocyte, proliferation and differentiation and the treatment or prophylaxis of those conditions using an E2F pathway modulator, and optionally a differentiation stimulus.

Bibliographic details of the publications numerically referred to in this specification are collected at the end of the description.

### BACKGROUND OF THE INVENTION

The major function of the skin is to act as a barrier between the internal and external environment. The skin is divided into two layers, the dermis and the epidermis, of which the major cell type in the epidermis is the keratinocyte. During the strictly regulated process of differentiation, keratinocytes undergo morphological and biochemical changes, resulting in dead, enucleated, flat cells that are eventually sloughed from the skin surface. This process of differentiation is initiated by the irreversible growth arrest and suppression of proliferation specific genes such as p53 (4), E2F1 (1, 5), cdk1 (6) and keratin 14 (7) in proliferative basal cells. Concomitant with the suppression of proliferation-specific genes, there is a corresponding induction of differentiation-specific genes, such as keratin 10 (8), cornifin (9) and transglutaminase type 1 (5). This process of growth suppression and induction of terminal differentiation is predominantly regulated at the transcriptional level by several transcription factor families such as AP1, Sp1, AP2 and E2F (2, 10, 11) and disruption of this process frequently accompanies the onset of neoplasia.

E2F was first identified as a nuclear factor capable of binding to the adenovirus E2 promoter (12). To date, six members of the E2F transcription factor family have been cloned, E2Fs 1-6 (13-25). E2F exists as a heterodimeric complex in association with a dimeric partner protein, DP1 or DP2 (26-28). This “free” E2F complex acts as a potent trans-activator of E2F-responsive genes. However, the activity of E2F is subject to regulation through inhibitory interactions with hypophosphorylated forms of the pocket proteins, pRb, p107 and p130 (14-19, 22, 29). Specifically, E2Fs 1-3 preferentially bind to pRb, whilst E2Fs 4-5 bind p107 and E2F5 binds p130. This direct association of E2F isoforms with their cognate pocket protein partne acts to repress E2F-mediated transcriptional activity. In some instances, this repression requires further interactions with specific histone deacetylases (30, 31). The presence of these various E2F:pocket protein complexes acts to regulate passage through various phases of the cell cycle. In particular, certain complexes are associated with a specific phase of the cell cycle: the E2F5:p130 complex associates with G0 (32), E2F1-3:pRb with G1 (33) and E2F4:p107 with G0/G1 phase (19). Thus, the coordinated activation/inactivation of these complexes illustrates that cell cycle progression is controlled by complex transcriptional means.

Recently, two new members of the E2F family, E2F6 and E2F7, have been characterized, which also repress E2F-mediated transcriptional activity, presumably by competing for binding to E2F responsive elements (e.g., E2F1 promoter, cdc2 promoter). In particular, E2F6 lacks residues for pocket binding or transactivation and is proposed to mediate E2F repression either through its direct binding to polycomb proteins or through the formation of a large multimeric complex containing Mga and Max proteins (69, 70). E2F7 possesses two distinct DNA binding domains and lacks a dimerisation domain as well as a transcriptional activation and a Rb-binding domain (71).

Despite clear evidence implicating E2F involvement in cell cycle regulation, there is also compelling evidence for other roles of E2F. For instance, E2Fs 1-3 have been implicated in the initiation of apoptosis (34-37). More recently, E2Fs have also been demonstrated to play a role in the regulation of myocyte, megakaryocyte and adipocyte differentiation (38-40).

In work leading up to the present invention, evidence was found that E2F1 may have a dual role as both a mediator of keratinocyte proliferation and a suppressor of squamous differentiation. If this were true, it would suggest that deregulated E2F in SCCs may serve to deregulate proliferation and repress terminal differentiation. Therefore, the present inventor examined the possibility that E2F may act as a biologically-relevant modulator of squamous differentiation and that E2F inhibition may provide the basis of a “differentiation therapy” for SCCs as well as other cancers. These studies revealed that overexpression of E2F isoforms in confluent primary keratinocyte cultures resulted in suppression of differentiation-associated markers. Moreover, they revealed that the DNA-binding domain and trans-activation domain of E2F1 are important in mediating suppression of differentiation. Use of a dominant/negative form of E2F1 (E2F d/n) found that E2F inhibition alone is sufficient to suppress the activity of proliferation-associated markers but is not capable of inducing differentiation markers. However, if the E2F d/n is expressed in differentiated keratinocytes, differentiation marker activity is further induced, suggesting that E2F may act as a modulator of squamous differentiation. The present inventor also examined the effects of E2F d/n in a differentiation-insensitive SCC cell line and found that treatment with the differentiating agent, 12-O-tetradecanoylphorbol-13-acetate (TPA), or expression of E2F d/n alone had no effect on differentiation markers. However, a combination of E2F d/n+TPA induced the expression of differentiation markers. Combined, these data broadly indicate that the E2F pathway plays a key role in keratinocyte differentiation and illustrate the unique potential of anti-E2F pathway therapies in arresting proliferation and inducing differentiation of SCCs.

### SUMMARY OF THE INVENTION

Accordingly, in one aspect, the present invention provides methods for modulating the proliferation and/or differentiation of an epithelial cell, especially a squamous epithelial cell, which is suitably selected from epidermis, oral mucosa, oesophageal, vaginal, tracheal or corneal epithelia. These methods generally comprise modulating an E2F pathway in the epithelial cell. Typically, the E2F pathway is modulated by modulating the level or functional activity of an expression product of a gene selected from an E2F gene or a gene belonging to the same regulatory pathway as the E2F gene (e.g., CycD, CycE, RepA, cdk2, cdk4, Rb, E2F1, cdk1, p107, thymidylate synthase, dihydrofolate reductase, c-myc, transglutaminase type 1, Sp1 or Sp3). In some embodiments, the expression product is an E2F transcript which suitably comprises a nucleotide sequence corresponding to any one of E2F1, E2F2, E2F3, E2F4, E2F5, E2F6 and E2F7. In other embodiments, the E2F expression product is an E2F polypeptide which suitably comprises an amino acid sequence corresponding to any one of E2F1, E2F2, E2F3, E2F4, E2F5, E2F6 and E2F7. Non limiting examples of suitable agents for modulating the E2F pathway include small molecules, such as nucleic acids, peptides, polypeptides (e.g., dominant negative polypeptides), peptidomimetics, carbohydrates, lipids or other organic (carbon-containing) or inorganic molecules, as further described herein. Suitably, the agent increases or reduces the level or functional activity of an expression product of a gene belonging to the E2F pathway by at least 10%, 20%, 30%, 40%, 50%, 60%, 70%, 80%, 90% relative to the level or funcational activity in the absence of the agent.

Suitably, the epithelial cell is a cancer cell, especially a skin cancer cell and more especially a squamous cell carcinoma cell. In some embodiments, the E2F pathway is antagonized to thereby inhibit or arrest the proliferation of the cell and to potentiate or induce its differentiation, e.g., by reducing or abrogating the level or functional activity of an expression product of an E2F activator gene or of a gene that is directly or indirectly modulated by an expression product of the E2F activator gene (e.g., CycD, CycE, RepA, cdk2, cdk4, Rb, E2F1, cdk1, p107, thymidylate synthase, dihydrofolate reductase, c-myc, transglutaminase type 1, Sp1 or Sp3) in the epithelial cell. Illustrative E2F activators include, but are not limited to, E2F1, E2F2 and E2F3. Illustrative inhibitors of E2F activators include, but are not restricted antisense oligonucleotide, ribozyme or RNAi molecule that selectively binds to a nucleic acid molecule that encodes an E2F activator, an intracellular E2F activator binding protein such as an antigen-binding molecule that is specifically immuno-interactive with an E2F activator or a polynucleotide from which that antigen-binding molecule is expressible, a dominant negative E2F activator polypeptide or a polynucleotide from which that dominant negative polypeptide is expressible as well as E2F6 and E2F7 polypeptides or their variants or derivatives and polynucleotides from which those polypeptides, variants or derivatives are expressible.

In other embodiments, the agent agonizes an E2F repressor to thereby inhibit or arrest the proliferation of the cell and to potentiate or induce its differentiation, e.g., by enhancing the level or functional activity of an expression product of an E2F repressor gene in the epithelial cell. Illustrative E2F repressors include, but are not limited to, E2F4, E2F5, E2F6 and E2F7. Exemplary agents that enhance the level or functional activity of E2F repressors include, but are not restricted to, polynucleotides from which E2F repressor genes are expressible and expression products of such genes.

Suitable agents that are useful in the above methods are characterized in that they bind to an expression product of a gene as broadly described above or to a genetic sequence (e.g., a transcriptional element) that modulates the expression of the gene, as determined by: contacting a preparation comprising at least a portion of an expression product of a gene as broadly described above, or a variant or derivative of the expression product, or a genetic sequence that modulates the expression of the gene, with the agent; and detecting a change in the level or functional activity of the at least a portion of the expression product, or the variant or derivative, or of a product expressed from the genetic sequence. For example, in embodiments in which an agent antagonizes an E2F pathway, the agent may bind to an E2F polypeptide or to a genetic sequence (e.g., a promoter or cis-acting sequence associated therewith) that modulates the expression of an E2F gene, as determined by: contacting a preparation comprising an E2F polypeptide or a biologically active fragment thereof, or a variant or derivative of these, or a genetic sequence that modulates the expression of an E2F gene; and detecting a decrease in the level or functional activity of the E2F polypeptide or biologically active fragment thereof, or variant or derivative, or of a product expressed from the genetic sequence.

In some embodiments, the method further comprises exposing the epithelial cell to a differentiation stimulus, which is suitably selected from high calcium concentrations, phorbol esters, butyric acid, activators of PPRγ-type receptors, macrocyclic diterpenes selected from compounds of the ingenane, pepluane and jatrophane families, indirubins, histone deacetylase inhibitors, retinoids and TGFβ1.

In another aspect, the invention provides compositions for modulating the proliferation and/or differentiation of an epithelial cell. These compositions generally comprise an agent that modulates an E2F pathway and a differentiation-stimulating agent that stimulates or otherwise induces the differentiation of an epithelial cell. Optionally, these compositions further comprise a pharmaceutically acceptable carrier.

In yet another aspect, the invention provides methods for treating or preventing a skin cancer or tumor of epithelial origin, especially squamous cell carcinoma, in a patient. These methods generally comprise administering to the patient an effective amount of an agent that antagonizes the function of an E2F pathway, as broadly described above. In some embodiments, the methods further comprise separately, sequentially or simultaneously administering a differentiation stimulus as broadly described above.

In still another aspect, the invention contemplates the use of an agent that antagonizes the function of an E2F pathway and optionally a differentiation-stimulating agent, as broadly described above, in the preparation of a medicament for the treating or preventing a skin cancer or tumor of epithelial origin, especially a squamous cell carcinoma.

In a further aspect, the present invention provides methods for identifying agents that modulate the proliferation and/or differentiation of an epithelial cell, especially a squamous epithelial cell, which is suitably selected from epidermis, oral mucosa, oesophageal, vaginal, tracheal or corneal epithelia. In some embodiments, these methods generally comprise identifying modulators (e.g., agonists or antagonists) of the E2F pathway, e.g., by contacting a preparation with a test agent, wherein the preparation comprises (i) a polypeptide comprising an amino acid sequence corresponding to at least a biologically active fragment of a polypeptide component of the E2F pathway, or to a variant or derivative thereof; or (ii) a polynucleotide comprising at least a portion of a genetic sequence that regulates the component, which is operably linked to a reporter gene. A detected change in the level or functional activity of the polypeptide component, or an expression product of the reporter gene, relative to a normal or reference level and/or functional activity in the absence of the test agent, indicates that the agent modulates the proliferation and/or differentiation of an epithelial cell.

In other embodiments, the methods generally comprise contacting a first sample of cells expressing an E2F pathway component and measuring a marker; contacting a second sample of cells expressing the component with an agent and measuring the marker; and comparing the marker of the first sample of cells with the marker of the second sample of cells. In various embodiments, these methods measure the levels of various components of the E2F pathway (e.g., intracellular components, illustrative examples of which include an expression product of the cdc2 gene and the E2F1 gene), or combinations of such markers, or combinations of one or more of such markers with markers associated with the proliferation and/or differentiation of epithelial cells.

In still other embodiments, the methods generally comprise administering to an animal model, or a human, an agent suspected of modulating the E2F pathway, and measuring the animal's responsiveness to the agent. In these embodiments, the method can be practiced with agents as described above and the animals or humans can be examined for the proliferation and/or differentiation of epithelial cells, especially of squamous epithelial cells.

In still another aspect, the present invention provides methods for diagnosing the presence, or risk of development, of a skin cancer or tumor of epithelial origin, especially a squamous cell carcinoma, in a test subject. These methods generally comprise detecting in the test subject aberrant expression of at least one gene selected from an E2F gene or a gene belonging to the same regulatory pathway as the E2F gene (e.g., CycD, CycE, RepA, cdk2, cdk4, Rb, E2F1, cdk1, p107, thymidylate synthase, dihydrofolate reductase, c-myc, transglutaminase type 1, Sp1 or Sp3), hereafter referred to an E2F pathway marker gene. Polynucleotide expression products of E2F pathway marker genes are referred to herein as “E2F pathway marker polynucleotides.” Polypeptide expression products of the E2F pathway marker genes are referred to herein as “E2F pathway marker polypeptides.”

Typically, such aberrant expression is detected by: (1) measuring in a biological sample obtained from the test subject the level or functional activity of an expression product of at least one E2F pathway marker gene and (2) comparing the measured level or functional activity of each expression product to the level or functional activity of a corresponding expression product in a reference sample obtained from one or more normal subjects or from one or more subjects lacking the skin cancer or tumor of epithelial origin, wherein a difference in the level or functional activity of the expression product in the biological sample as compared to the level or functional activity of the corresponding expression product in the reference sample is indicative of the presence, or risk of development, of the skin cancer or tumor of epithelial origin in the test subject.

In some embodiments, the methods comprise detecting increased expression of an E2F pathway marker gene selected from the group consisting of E2F1, E2F2 and E2F3. In specific embodiments, the methods comprise detecting increased expression of E2F1.

In some embodiments, the methods further comprise diagnosing the presence, stage, degree, or risk of development, of the skin cancer or tumor of epithelial origin in the test subject when the measured level or functional activity of the or each expression product is different than the measured level or functional activity of the or each corresponding expression product. In these embodiments, the difference typically represents an at least about 10%, 20%, 30%, 40%, 50%, 60%, 70%, 80% or 90%, or even an at least about 100%, 200%, 300%, 400%, 500%, 600%, 700%, 800%, 900% or 1000% increase, or an at least about 10%, 20%, 30% 40%, 50%, 60%, 70%, 80%, 90%, 92%, 94%, 96%, 97%, 98% or 99%, or even an at least about 99.5%, 99.9%, 99.95%, 99.99%, 99.995% or 99.999% decrease in the level or functional activity of an individual expression product as compared to the level or functional activity of an individual corresponding expression product. In some embodiments, the method further comprises diagnosing the absence of the skin cancer or tumor of epithelial origin or the absence of risk of developing the skin cancer or tumor of epithelial origin in the test subject when the measured level or functional activity of the or each expression product is the same as or similar to the measured level or functional activity of the or each corresponding expression product. In these embodiments, the measured level or functional activity of an individual expression product varies from the measured level or functional activity of an individual corresponding expression product by no more than about 20%, 18%, 16%, 14%, 12%, 10%, 9%, 8%, 7%, 6%, 5%, 4%, 3%, 2%, 1%, or 0.1%.

In some embodiments, the methods comprise measuring the level or functional activity of individual expression products of at least about 2, 3, 4 or 5 E2F pathway marker genes. For example, the methods may comprise measuring the level or functional activity of an E2F pathway marker polynucleotide either alone or in combination with as much as 4, 3, 2 or 1 other E2F pathway marker polynucleotide(s). In another example, the methods may comprise measuring the level or functional activity of an E2F pathway marker polypeptide either alone or in combination with as much as 4, 3, 2 or 1 other E2F pathway marker polypeptides(s).

Advantageously, the biological sample is a tissue biopsy, which suitably contains an epithelial cell, especially a squamous epithelial cell, such as from epidermis, oral mucosa, oesophageal, vaginal, tracheal or corneal epithelia. Typically, the expression product is selected from a RNA molecule or a polypeptide. In some embodiments, the expression product is the same as the corresponding expression product. In other embodiments, the expression product is a variant (e.g., an allelic variant) of the corresponding expression product.

In certain embodiments, the expression product or corresponding expression product is a target RNA (e.g., mRNA) or a DNA copy of the target RNA whose level is measured using at least one nucleic acid probe that hybridizes under at least low stringency conditions to the target RNA or to the DNA copy, wherein the nucleic acid probe comprises at least 15 contiguous nucleotides of an E2F pathway marker gene. In these embodiments, the measured level or abundance of the target RNA or its DNA copy is normalized to the level or abundance of a reference RNA or a DNA copy of the reference RNA that is present in the same sample. Suitably, the nucleic acid probe is immobilized on a solid or semi-solid support. In illustrative examples of this type, the nucleic acid probe forms part of a spatial array of nucleic acid probes. In some embodiments, the level of nucleic acid probe that is bound to the target RNA or to the DNA copy is measured by hybridisation (e.g., using a nucleic acid array). In other embodiments, the level of nucleic acid probe that is bound to the target RNA or to the DNA copy is measured by nucleic acid amplification (e.g., using a polymerase chain reaction (PCR)). In still other embodiments, the level of nucleic acid probe that is bound to the target RNA or to the DNA copy is measured by nuclease protection assay.

In other embodiments, the expression product or corresponding expression product is a target polypeptide whose level is measured using at least one antigen-binding molecule that is immuno-interactive with the target polypeptide. In these embodiments, the measured level of the target polypeptide is normalized to the level of a reference polypeptide that is present in the same sample. Suitably, the antigen-binding molecule is immobilized on a solid or semi-solid support. In illustrative examples of this type, the antigen-binding molecule forms part of a spatial array of antigen-binding molecule. In some embodiments, the level of antigen-binding molecule that is bound to the target polypeptide is measured by immunoassay (e.g., using an ELISA).

### BRIEF DESCRIPTION OF THE DRAWINGS

FIG. 1 is a graphical representation showing that E2F1 suppresses differentiation markers in primary human keratinocyte cultures. Human epidermal keratinocytes were transfected with a luciferase reporter linked to the (A) transglutaminase-l promoter (TG-1 Luc) or (B) keratin-10 promoter (K10 Luc). Each construct was co-transfected with a ¥â actin-CAT reporter, to correct for transfection efficiency, as well as either an E2F1 expression plasmid or GFP expression plasmid. Cells were transfected when proliferating (Prol) or differentiated (Conf). Cells were induced to differentiate by growth to confluence or treatment of cells with media supplemented with 50ng/mL of 12-O-tetradecanoylphorbol-13-acetate (TTIA) for 48 h. Data presented as mean±SEM of triplicate determinations of at least three experiments (*=p<0.05 compared to prol).

FIG. 2 is a graphical representation showing that E2Fs 1-5 suppress TG-1 Luc in confluent cultures of primary keratinocytes. Confluent cultures of human epidermal keratinocytes were transfected with a luciferase reporter linked to the transglutaminase-1 promoter (TG-1 Luc). Each construct was co-transfected with a β-actin-CAT reporter, to correct for transfection efficiency, as well as E2F expression plasmids (E2F 1, E2F2, E2F3, E2F4 or E2F5) or GFP as control. Data presented as mean±SEM of triplicate determinations of at least three experiments and are normalized such that the value of the control is 100 (*=p<0.05 compared to control). CBD, cyclin binding domain; DBD, DNA binding domain; HDD, heterodimerisation domain; TD, trans-activation domain and PPBD, pocket protein binding domain.

FIG. 3 is a graphical representation showing that E2F1 suppression of TG-1 Luc requires the DNA binding domain, transactivation domain and pocket protein binding domain. (A) Proliferating (Prol) and (B) differentiated (Conf) keratinocytes were transfected with a reporter linked to either the (A) cdc2 promoter (cdc2-CAT) or (B) transglutaminase-1 promoter (TG-1 Luc). Each plasmid was co-transfected with a-actin-CAT or β-actin-Luc reporter, to normalize for transfection efficiency, as well as an expression plasmid for one of the following: E2Fd/n, E2F1, Δ132E2F1, Δ409 E2F1 or GFP as control. Data presented as mean±SEM of triplicate determinations of at least three experiments (*=p<0.05 compared to prol (A) or conf (B)). CBD, cyclin binding domain; DBD, DNA binding domain; HDD, heterodimerisation domain; TD, trans-activation domain and PPBD, pocket protein binding domain.

FIG. 4 is a graphical representation showing that E2F modulates squamous differentiation (A) Proliferating and (B, C) differentiated keratinocytes were transfected with a reporter linked to the (A) cdc2 promoter (cdc2-CAT), (B) transglutaminase-1 promoter (TG-1 Luc) or (C) keratin 10 promoter (K10-Luc). Each condition was transfected with a β-actin-CAT or β-actin-Luc reporter, to normalize for transfection efficiency, as well as E2Fd/n or pCDNA-GFP as control. Data presented as mean±SEM of triplicate determinations of at least three experiments (*=p<0.05 compared to control).

FIG. 5 is a photographic representation showing that E2Fs 1-5 are expressed in primary human keratinocytes. Proliferative (P) and differentiated (D) keratinocytes were harvested, total cellular protein isolated and 5 μg protein subjected to Western blot analysis for the expression of E2Fs 1-5.

FIG. 6 is a graphical representation showing that E2F inhibition makes KJD-1/SV40 cells permissive to TPA-mediated differentiation. Proliferating KJD-1/SV40 cells were transfected with a luciferase reporter linked to the transglutaminase-1 promoter (TG-1 Luc). Cells were co-transfected with a β-actin-CAT reporter, to adjust for transfection efficiency, and either the E2Fd/n plasmid or pCDNA-GFP control (pCDNA). After transfection, cells remained in growth media or were treated with 50 ng/ml of 12-O-tetradecanoylphorbol-ester (TPA) for 48 hours. Data are presented as mean ᵢ¾ SEM of triplicate determinations of at least three experiments and normalized such that the value of the control is 100 (*=p<0.05 compared to control).

FIG. 7 is a schematic representation of a model illustrating the requirement for independent stimuli to induce growth inhibition or differentiation in keratinocytes.

### BRIEF DESCRIPTION OF THE SEQUENCES: SUMMARY TABLE

TABLE A

| SEQUENCE ID | | |
|---|---|---|
| NUMBER | SEQUENCE | LENGTH |
| SEQ ID NO: 1 | Nucleotide sequence corresponding to the E2F1 cDNA | 2486 bases |
| | molecule disclosed in GenBank Accession No. NM_005225 | |
| SEQ ID NO: 2 | Amino acid sequence corresponding to E2F1 as disclosed in | 437 residues |
| | GenPept Accession No. NM_005216 | |
| SEQ ID NO: 3 | Nucleotide sequence corresponding to the E2F2 cDNA | 1766 bases |
| | molecule disclosed in GenBank Accession No. NM_004091 | |
| SEQ ID NO: 4 | Amino acid sequence corresponding to E2F2 as disclosed in | 437 residues |
| | GenPept Accession No. NM_004082 | |
| SEQ ID NO: 5 | Nucleotide sequence corresponding to the E2F3 cDNA | 4744 bases |
| | molecule disclosed in GenBank Accession No. AF547386 | |
| SEQ ID NO: 6 | Amino acid sequence corresponding to E2F3 as disclosed in | 465 residues |
| | GenPept Accession No. NP_001940 | |
| SEQ ID NO: 7 | Nucleotide sequence corresponding to the E2F4 cDNA | 1322 bases |
| | molecule disclosed in GenBank Accession No. NM_001950 | |
| SEQ ID NO: 8 | Amino acid sequence corresponding to E2F4 as disclosed in | 413 residues |
| | GenPept Accession No. NP_001941 | |
| SEQ ID NO: 9 | Nucleotide sequence corresponding to the E2F5 cDNA | 1752 bases |
| | molecule disclosed in GenBank Accession No. AY162833 | |
| SEQ ID NO: 10 | Amino acid sequence corresponding to E2F5 as disclosed in | 346 residues |
| | GenPept Accession No. NP_001942 | |
| SEQ ID NO: 11 | Nucleotide sequence corresponding to the E2F6 cDNA | 2043 bases |
| | molecule disclosed in GenBank Accession No. NM_001952 | |
| SEQ ID NO: 12 | Amino acid sequence corresponding to E2F6 as disclosed in | 281 residues |
| | GenPept Accession No. NP_001943 | |
| SEQ ID NO: 13 | Nucleotide sequence corresponding to the E2F7 cDNA | 3624 bases |
| | molecule disclosed in GenBank Accession No. XM_196008 | |
| SEQ ID NO: 14 | Amino acid sequence corresponding to E2F7, which is | 908 residues |
| | encoded by the nucleotide sequence set forth in SEQ ID | |
| | NO: 13 | |
| SEQ ID NO: 15 | Nucleotide sequence corresponding to E2F1 dominant | 366 bases |
| | negative mutant 1 cDNA | |
| SEQ ID NO: 16 | Amino acid sequence corresponding to E2F1 dominant | 121 residues |
| | negative mutant 1 | |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

“Antigenic or immunogenic activity” refers to the ability of a polypeptide, fragment, variant or derivative according to the invention to produce an antigenic or immunogenic response in an animal, suitably a mammal, to which it is administered, wherein the response includes the production of elements which specifically bind the polypeptide or fragment thereof.

By “biologically active fragment” is meant a fragment of a full-length parent polypeptide which fragment retains an activity of the parent polypeptide. As used herein, the term “biologically active fragment” includes deletion variants and small peptides, for example of at least 6, 7, 8, 9, 10, 11, 12, 13, 14, 15, 16, 17, 18, 19, 20, 21, 22, 23, 24, 25, 26, 27, 28, 29, 30, 35, 40, 45, 50 contiguous amino acid residues, which comprise an activity of the parent polypeptide. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

The term “biological sample” as used herein refers to a sample that may extracted, untreated, treated, diluted or concentrated from a patient. Suitably, the biological sample is a tissue biopsy, more preferably from an epithelial cell, especially a squamous epithelial cell such as from epidermis, oral mucosa, oesophageal, vaginal, tracheal or corneal epithelia.

As used herein, the term “cis-acting sequence” or “cis-regulatory region” or similar term shall be taken to mean any sequence of nucleotides, which when positioned appropriately relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of any structural gene sequence at the transcriptional or post-transcriptional level.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions or deletions that provide for functional equivalent molecules.

The term “differentiation potential” as used herein means the capacity of a cancer cell, especially a squamous cell carcinoma cell, to respond, or the magnitude of the response, to a signal which promotes its functional maturation into a normal cell, especially a normal cell of the keratinocyte lineage. An “increase in differentiation potential” may be seen to be conferred by a test molecule wherein, for example, a co-culture of cancer cells with the test molecule for a sufficient time and under appropriate conditions results in an increase in the response of the cancer cells to a differentiation-inducing agent, which may be observed inter alia as a rise in the number of cancer cells undergoing differentiation or an increase in the rate at which the cancer cells undergo differentiation.

“Differentiation” refers to the developmental process whereby cells assume a specialized phenotype, i.e., acquire one or more characteristics or functions distinct from other cell types. Typically, differentiation is a staged process, e.g., in development, through which a cell progressively acquires distinguishably new phenotypic attributes. In most uses, the differentiated phenotype refers to a cell phenotype that is at the mature endpoint in some developmental pathway. In many but not all tissues, the process of differentiation is coupled with exit from the cell cycle—in these cases, the cells lose or greatly restrict their capacity to proliferate when they differentiate.

A “dominant negative” polypeptide is an inactive variant of a polypeptide, which, by interacting with the cellular machinery, displaces an active polypeptide from its interaction with the cellular machinery or competes with the active polypeptide, thereby reducing the effect of the active polypeptide. For example, a dominant negative transcription factor which binds to a promoter site in the control region of a gene but does not increase gene transcription can reduce the effect of a normal transcription factor by occupying promoter binding sites without increasing transcription. Alternatively, a dominant negative transcription factor, which binds to a promoter site in the control region of a gene, and displaces the binding of the normal transcription factor to the promoter site could cause an increase in transcription if the normal transcription factor it displaces was involved in active repression of the control region of the gene. For instance, if the normal transcription factor (e.g:, E2F) existed in a complex with corepressors such as hypophosphorylated Rb and/or histone deacetylase enzymes which were bound to the control region of the gene then one would predict that displacement of this repressor complex could lead to derepression/activation of the control region of the gene. Likewise a dominant negative receptor which binds a ligand but does not transmit a signal in response to binding of the ligand can reduce the biological effect of expression of the ligand. Similarly, a dominant negative catalytically-inactive kinase which interacts normally with target proteins but does not phosphorylate the target proteins can reduce phosphorylation of the target proteins in response to a cellular signal. The end result of the expression of a dominant negative polypeptide in a cell is a reduction in function of active proteins. One of ordinary skill in the art can assess the potential for a dominant negative variant of a protein, and using standard mutagenesis techniques to create one or more dominant negative variant polypeptides.

By “effective amount”, in the context of modulating an activity or of treating or preventing a condition is meant the administration of that amount of active ingredient to an individual in need of such modulation, treatment or prophylaxis, either in a single dose or as part of a series, that is effective for modulation of that effect or for treatment or prophylaxis or improvement of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the term “function” refers to a biological, enzymatic, or therapeutic function.

The term “gene” as used herein refers to any and all discrete coding regions of the cell's genome, as well as associated non-coding and regulatory regions. The gene is also intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The DNA sequences may be cDNA or genomic DNA or a fragment thereof. The gene may be introduced into an appropriate vector for extrachromosomal maintenance or for integration into the host.

“Hybridisation” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

Reference herein to “immuno-interactive” includes reference to any interaction, reaction, or other form of association between molecules and in particular where one of the molecules is, or mimics, a component of the immune system.

By “isolated” is meant material that is substantially or essentially free from components that normally accompany it in its native state.

By “modulating” is meant increasing or decreasing, either directly or indirectly, the level or functional activity of a target molecule. For example, an agent may indirectly modulate the level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

The term “5′ non-coding region” is used herein in its broadest context to include all nucleotide sequences which are derived from the upstream region of an expressible gene, other than those sequences which encode amino acid residues which comprise the polypeptide product of said gene, wherein 5′ non-coding region confers or activates or otherwise facilitates, at least in part, expression of the gene.

By “obtained from” is meant that a sample such as, for example, a polynucleotide extract or polypeptide extract is isolated from, or derived from, a particular source of the host. For example, the extract can be obtained from a tissue or a biological fluid isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

The term “operably connected” or “operably linked” as used herein means placing a structural gene under the regulatory control of a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e. the genes from which it is derived.

The term “patient” refers to patients of human or other animal origin and includes any individual it is desired to examine or treat using the methods of the invention. However, it will be understood that “patient” does not imply that symptoms are present. Suitable animals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes, avians, reptiles).

By “pharmaceutically acceptable carrier” is meant a solid or liquid filler, diluent or encapsulating substance that can be safely used in topical or systemic administration to a mammal.

The term “polynucleotide” or “nucleic acid” as used herein designates mRNA, RNA, cRNA, cDNA or DNA. The term typically refers to oligonucleotides greater than 30 nucleotide residues in length.

The terms “polynucleotide variant” and “variant” refer to polynucleotides displaying substantial sequence identity with a reference polynucleotide sequence or polynucleotides that hybridize with a reference sequence under stringent conditions as known in the art (see for example Sambrook et al., Molecular Cloning. A Laboratory Manual”, Cold Spring Harbor Press, 1989). These terms also encompass polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains a biological function or activity of the reference polynucleotide. The terms “polynucleotide variant” and “variant” also include naturally-occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally occurring amino acid polymers.

The term “polypeptide variant” refers to polypeptides in which one or more amino acids have been replaced by different amino acids. It is well understood in the art that some amino acids may be changed to others with broadly similar properties without changing the nature of the activity of the polypeptide (conservative substitutions) as described hereinafter. These terms also encompass polypeptides in which one or more amino acids have been added or deleted, or replaced with different amino acids.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but can alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Preferably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridisation conditions. Probes can be labelled directly or indirectly.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e. upstream activating sequences, enhancers and silencers) which alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene. Preferred promoters according to the invention may contain additional copies of one or more specific regulatory elements to further enhance expression in a cell, and/or to alter the timing of expression of a structural gene to which it is operably connected.

The term “recombinant polynucleotide” as used herein refers to a polynucleotide formed in vitro by the manipulation of a polynucleotide into a form not normally found in nature. For example, the recombinant polynucleotide can be in the form of an expression vector. Generally, such expression vectors include transcriptional and translational regulatory polynucleotide operably linked to the polynucleotide.

By “recombinant polypeptide” is meant a polypeptide made using recombinant techniques, i.e., through the expression of a recombinant or synthetic polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

The term “synthetic polynucleotide” as used herein refers to a polynucleotide formed in vitro by the manipulation of a polynucleotide into a form not normally found in nature. For example, the synthetic polynucleotide can be in the form of an expression vector. Generally, such expression vectors include transcriptional and translational regulatory polynucleotide operably linked to the polynucleotide.

The term “synonymous codon” as used herein refers to a codon having a different nucleotide sequence than another codon but encoding the same amino acid as that other codon.

By “translational efficiency”, is meant the efficiency of a cell's protein synthesis machinery to incorporate the amino acid encoded by a codon into a nascent polypeptide chain. This efficiency can be evidenced, for example, by the rate at which the cell is able to synthesize the polypeptide from an RNA template comprising the codon, or by the amount of the polypeptide synthesized from such a template.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticing) and the hph gene which confers resistance to the antibiotic hygromycin B.

The terms “wild-type” and “normal” are used interchangeably to refer to the phenotype that is characteristic of most of the members of the species occurring naturally and contrast for example with the phenotype of a mutant.

As used herein, underscoring or italicising the name of a gene shall indicate the gene, in contrast to its protein product, which is indicated by the name of the gene in the absence of any underscoring or italicising. For example, “E2F1” shall mean the E2F1 gene or transcript thereof, whereas “E2F1” shall indicate the protein product or products generated from transcription and translation and alternative splicing of the “E2F1” gene.

2. Method of Modulating the Proliferation and/or Differentiation of Epithelial Cells

The present invention is predicated in part on the discovery that overexpression of E2F isoforms in confluent primary keratinocyte cultures results in suppression of differentiation-associated markers and that the DNA binding domain and trans-activation domain of these isoforms, especially of E2F1, are important in mediating this suppression. The present inventor also found that E2F inhibition suppresses proliferation markers in primary keratinocytes and ‘potentiates’, or increases potential for, their differentiation (i.e., a “priming” effect). It has also been discovered that the pro-proliferative effect of E2F is abrogated by an E2F d/n, especially an E2F1 d/n. It is proposed, therefore, that modulators of an E2F pathway will be useful inter alia for the treatment or prevention of a skin cancer or tumor of epithelial origin, especially squamous cell carcinoma.

Accordingly, the present invention provides methods for modulating the proliferation and/or differentiation of an epithelial cell, especially a squamous epithelial cell, comprising contacting the cell with an agent for a time and under conditions sufficient to modulate an E2F pathway, especially an E2F 1 pathway, including modulating the expression of a gene or the level and/ or functional activity of an expression product of that gene, wherein the gene is selected from an E2F gene, a gene relating to the same regulatory or biosynthetic pathway as the E2F gene, or a gene whose expression product modulates (e.g., promotes, enhances or capacitates; or inhibits or impairs) the expression of the E2F gene, or a gene whose expression is modulated directly or indirectly by an expression product of the E2F gene. Representative members of an E2F pathway include (CycD, CycE, RepA, cdk2, cdk4, Rb, E2F1, cdk1, p107, thymidylate synthase, dihydrofolate reductase, c-myc, transglutaminase type 1, Sp1 or Sp3). In some embodiments, the epithelial cell is from a tissue or organ selected from epidermis, oral mucosa, oesophageal, vaginal, tracheal or corneal epithelia. In certain embodiments, the epithelial cell is a cancer cell, especially a skin cancer cell and more especially a squamous cancer cell.

In some embodiments, the E2F pathway is modulated by modulating the level or functional activity of an E2F expression product. Representative E2F members include, but are not limited to, E2F1, E2F2, E2F3, E2F4, E2F5, E2F6 and E2F7 and their variants, including splice variants. In some embodiments, proliferation of the epithelial cell is decreased or abrogated, and its differentiation is induced or potentiated, by reducing or abrogating the expression of an E2F activator gene or the level or functional activity of its expression product or by enhancing or reducing the expression of the regulator gene or the level or functional activity of its expression product, depending upon whether it is a repressor or activator of the E2F activator gene or its expression product, respectively. Illustrative E2F activators include, but are not limited to, E2F1, E2F2 and E2F3. Representative nucleic acid and amino acid sequences for these activators are set forth in SEQ ID NO:1-6.

Suitable agents for reducing or abrogating gene expression include, but are not restricted to, oligoribonucleotide sequences, including anti-sense RNA and DNA molecules and ribozymes, that function to inhibit the translation, for example, of E2F- or mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions are preferred.

Ribozymes are enzymatic RNA molecules capable of catalysing the specific cleavage of RNA. The mechanism of ribozyme action involves sequence specific hybridisation of the ribozyme molecule to complementary target RNA, followed by a endonucleolytic cleavage. Within the scope of the invention are engineered hammerhead motif ribozyme molecules that specifically and efficiently catalyse endonucleolytic cleavage of target sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridisation with complementary oligonucleotides, using ribonuclease protection assays.

Both anti-sense RNA and DNA molecules and ribozymes may be prepared by any method known in the art for the synthesis of RNA molecules. These include techniques for chemically synthesising oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include but are not limited to the addition of flanking sequences of ribo- or deoxy- nucleotides to the 5′ or 3′ ends of the molecule or the use of phosphorothioate or 2′ O-methyl rather than phosphodiesterase linkages within the oligodeoxyribonucleotide backbone.

Alternatively, RNA molecules that mediate RNA interference (RNAi) of a target gene or gene transcript can be used to reduce or abrogate gene expression. RNAi refers to interference with or destruction of the product of a target gene by introducing a single stranded, and typically a double stranded RNA (dsRNA) that is homologous to the transcript of a target gene. Thus, in some embodiments, dsRNA per se and especially dsRNA-producing constructs corresponding to at least a portion of a target gene may be used to reduce or abrogate its expression. RNAi-mediated inhibition of gene expression may be accomplished using any of the techniques reported in the art, for instance by transfecting a nucleic acid construct encoding a stem-loop or hairpin RNA structure into the genome of the target cell, or by expressing a transfected nucleic acid construct having homology for a target gene from between convergent promoters, or as a head to head or tail to tail duplication from behind a single promoter. Any similar construct may be used so long as it produces a single RNA having the ability to fold back on itself and produce a dsRNA, or so long as it produces two separate RNA transcripts which then anneal to form a dsRNA having homology to a target gene.

Absolute homology is not required for RNAi, with a lower threshold being described at about 85% homology for a dsRNA of about 200 base pairs (Plasterk and Ketting, 2000, Current Opinion in Genetics and Dev. 10: 562-67). Therefore, depending on the length of the dsRNA, the RNAi-encoding nucleic acids can vary in the level of homology they contain toward the target gene transcript, i.e., with dsRNAs of 100 to 200 base pairs having at least about 85% homology with the target gene, and longer dsRNAs, i.e., 300 to 100 base pairs, having at least about 75% homology to the target gene. RNA-encoding constructs that express a single RNA transcript designed to anneal to a separately expressed RNA, or single constructs expressing separate transcripts from convergent promoters, are preferably at least about 100 nucleotides in length. RNA-encoding constructs that express a single RNA designed to form a dsRNA via internal folding are preferably at least about 200 nucleotides in length.

The promoter used to express the dsRNA-forming construct may be any type of promoter if the resulting dsRNA is specific for a gene product in the cell lineage targeted for destruction. Alternatively, the promoter may be lineage specific in that it is only expressed in cells of a particular development lineage. For example, epithelial-specific promoters can be used in this regard including, but are not restricted to, a loricrin gene promoter and a keratin gene promoter (e.g., K10, K14 keratin gene promoter). This might be advantageous where some overlap in homology is observed with a gene that is expressed in a non-targeted cell lineage. The promoter may also be inducible by externally controlled factors, or by intracellular environmental factors.

In other embodiments, RNA molecules of about 21 to about 23 nucleotides, which direct cleavage of specific mRNA to which they correspond, as for example described by Tuschl et al. in U.S. patent application No. 20020086356, can be utilized for mediating RNAi. Such 21-23 nt RNA molecules can comprise a 3′ hydroxyl group, can be single-stranded or double stranded (as two 21-23 nt RNAs) wherein the dsRNA molecules can be blunt ended or comprise overhanging ends (e.g., 5′, 3′).

In still other embodiments, the E2F pathway is antagonized using a protein that binds to and renders an E2F pathway component non functional. Exemplary proteins of this type include anti-E2F antigen-binding molecules (e.g., a neutralising antibody), which are suitably produced with an expression vector that is introduced into the cell. Illustrative antigen-binding molecule include whole polyclonal or monoclonal antibodies, which can be prepared according to standard methods known to those skilled in the art. Exemplary protocols which may be used are described for example in Coligan et al., “Current Protocols In Immunology”, (John Wiley & Sons, Inc, 1991), and Ausubel et al., (1994-1998, supra), in particular Section III of Chapter 11. The invention also contemplates as antigen-binding molecules Fv, Fab, Fab′ and F(ab′)₂ immunoglobulin fragments. Alternatively, the antigen-binding molecule may be in the form of a synthetic stabilized Fv fragment, a single variable region domain (also known as a dAbs), a “minibody” and the like as known in the art. Also contemplated as antigen binding molecules are humanized antibodies. Techniques for producing humanized monoclonal antibodies are described, for example, by Jones et al. (1986, Nature 321:522), Carter et al. (1992, Proc. Natl. Acad. Sci. USA 89: 4285), Sandhu (1992, Crit. Rev. Biotech. 12: 437), Singer et al. (1993, J. Immun. 150: 2844), Sudhir (ed., Antibody Engineering Protocols, Humana Press, Inc. 1995), Kelley (“Engineering Therapeutic Antibodies”, in Protein Engineering: Principles and Practice Cleland et al. (eds.), pages 399-434 (John Wiley & Sons, Inc. 1996), and by Queen et al., U.S. Pat. No. 5,693,762 (1997).

In still other embodiments, the E2F pathway is antagonized using a compound that inhibits the activity of an E2F polypeptide. Representative compounds of this type include: E2F repressors such as but not limited to E2F6 and E2F7; as well as dominant negative (d/n) E2F polypeptides and small molecule modulators of E2F and E2F inhibitory peptides which can be identified, for example, using methods described in Section 3 infra.

E2F6 and E2F7 inhibit the transactivation of E2F responsive elements (e.g., E2F1 and cdc2 promoters) and are suitable, therefore, for antagonising the E2F pathway. These ‘E2F repressors’ or their variant and derivatives can be prepared using standard synthetic or recombinant techniques having regard to their nucleic acid and amino acid sequences set forth in SEQ ID NO:11-14.

E2F d/n polypeptides can be any E2F mutant polypeptides which inhibit or counteract the activity of wild-type E2F polypeptide activity. Suitably, a d/n E2F results from the functional inactivation of at least one domain of an E2F polypeptide. Representative E2F domains include, but are not limited to, the cyclin-binding domain, the DNA-binding domain, the transcription factor E2F/dimerisation partner (TDP) domain, the leucine zipper domain, the heterodimerisation domain, the trans-activation domain and the pocket protein binding domain. In some embodiments, the d/n E2F comprises at least one E2F domain, especially at least two E2F domains, and more especially at least three E2F domains, which is/are functionally inactivated. These domains are suitably selected from the cyclin binding domain, the pocket protein binding domain and the trans-activation domain. Typically, the E2F domains are functionally inactivated using standard mutagenesis techniques including, but not restricted to, site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. In some embodiments, the d/n E2F comprises one and desirably two functional domains selected from the DNA-binding domain and the heterodimerisation domain, or biologically active fragments thereof. A d/n E2F polypeptide can be expressed and purified exogenously or produced by an expression construct in the cell or tumor to be treated. A description of one example of a d/n E2F construct is provided in Example 4. In certain embodiments, the E2F d/n is produced in a tumor of epithelial origin using an expression vector, which comprises an E2F d/n-encoding polynucleotide operably linked to a regulatory polynucleotide (e.g., a transcriptional control element). In these embodiments, the E2F d/n-encoding polynucleotide is codon modified using codons that have a higher translational efficiency in the tumor cells than in corresponding normal epithelial cells, as for example described in Section 4 infra.

In accordance with the present invention, antagonising the function of an E2F pathway in a cell of epithelial origin markedly increases the cell's potential for subsequent differentiation. Thus, in certain embodiments, the method further comprises exposing the epithelial cell to a differentiation stimulus for a time and under condition sufficient for the epithelial cell to differentiate into a cell with a different phenotype, e.g., a terminally differentiated epithelial cell. This exposure is suitably carried out simultaneously or sequentially with contacting the cell with the E2F pathway antagonist. The differentiation stimulus is suitably selected from: high calcium concentrations; phorbol esters such as 12-O-tetradecanoylphorbol-13-acetate; butyric acid; activators of PPRγ-type receptors such as 5-{4-[2-(methyl-pyrid-2-ylamino)ethoxy]benzl}thiazolidine-2,4-dione, 3-{4-[2-(benzoxazol-2-ylmethylamino)ethoxy]phenyl}-2-ethoxypropionic acid, (+)-3-{4-[2-(benzoxazol-2-ylmethylamino)ethoxy]phenyl}-2-ethoxypropionic acid, and (−)-3-{4-[2-(benzoxazol-2-ylmethylamino)ethoxy]phenyl}-2-ethoxypropionic acid; indirubins such as meisoindigo; macrocyclic diterpenes selected from compounds of the ingenane, pepluane and jatrophane families as disclosed, for example, in WO01/93885; histone deacetylase inhibitors, retinoids and TGFβ1.

The agents of the invention will suitably modulate the proliferation of epithelial cells and induce or potentiate their differentiation. Assays measuring cell proliferation or differentiation are well known in the art. The ability of modulatory agents to stimulate or inhibit differentiation or proliferation of mammary cells can be measured using cultured epithelial cells, or in vivo by administering molecules of the present invention to the appropriate animal model. Generally, for cell proliferation, cell number is determined, directly, by microscopic or electronic enumeration, or indirectly, by the use of chromogenic dyes, incorporation of radioactive precursors or measurement of metabolic activity of cellular enzymes. For example, assays measuring proliferation include such assays as chemosensitivity to neutral red dye (Cavanaugh et al., 1990, Investigational New Drugs 8: 347-354), incorporation of radiolabelled nucleotides (Cook et al., 1989, Anal. Biochem. 179: 1-7), incorporation of 5-bromo-2′-deoxyuridine (BrdU) in the DNA of proliferating cells (Porstmann et al., 1985, J. Immunol. Methods 82: 169-79), and use of tetrazolium salts (Mosmann, 1983, J. Immunol. Methods 65 55-63; Alley et al., 1988, Cancer Res. 48: 589-601; Marshall et al., 1995, Growth Reg. 5: 69-84; and Scudiero et al., Cancer Res. 1988, 48: 4827-33) and by measuring proliferation using ³H-thymidine uptake (Crowley et al., 1990, J. Immunol. Meth. 133: 55-66). An exemplary cell proliferation assay comprises culturing cells in the presence and absence of a test compound, and detecting cell proliferation by, for example, measuring incorporation of BrdU.

Assays that measure differentiation include, for example, measuring cell-surface markers associated with epithelial cell stage-specific expression, enzymatic activity, functional activity or morphological changes (Watt, 1991, FASEB 5: 281-4; Francis, 1994, Differentiation 57: 63-75; Raes, 1989, Adv. Anim. Cell Biol. Technol. Bioprocesses, 161-71). Suitable differentiation markers include, for example, transglutaminase type 1, cytokeratin 10, T-Ag, involucrin, filaggrin, loricrin, vimentin and the keratins K4, K7, K8, K10/1, K13, K14, K17, K18 and K19, whose expression can be easily detected, for example, by means of generally obtainable antibodies.

In vivo assays, well known in the art, are available for evaluating the effect of modulatory agents on epithelial tissue. For instance, epithelial tissue (e.g., epidermis, dermis etc) is contacted with an agent or with a composition comprising the agent, for a time and under conditions to permit entry of the agent into the tissue. After a specific time duration sufficient for the agent to modulate the E2F pathway, animals are sacrificed and the tissue is removed and weighed. The tissue samples are then processed to produce cell suspensions whose proliferation rate and stage of differentiation are determined by standard methods, as described, for example, above.

3. Identification of Target Molecule Modulators

The invention also features methods of screening for agents that modulate an E2F pathway. In some embodiments, the methods comprise: (1) contacting a preparation with a test agent, wherein the preparation contains (i) a polypeptide comprising an amino acid sequence corresponding to at least a biologically active fragment of a polypeptide component of the E2F pathway, or to a variant or derivative thereof; or (ii) a polynucleotide comprising at least a portion of a genetic sequence that regulates the component, which is operably linked to a reporter gene; and (2) detecting a change in the level and/or functional activity of the polypeptide component, or an expression product of the reporter gene, relative to a normal or reference level and/or functional activity in the absence of the test agent, which indicates that the agent modulates the E2F pathway.

Any suitable assay for detecting, measuring or otherwise determining modulation of the E2F pathway (e.g., such as by detecting epithelial proliferation and differentiation potential), is contemplated by the present invention. Assays of a suitable nature are known to persons of skill in the art and examples of these are described in Section 2 supra.

Candidate modulatory agents encompass numerous chemical classes, including organic molecules such as small organic compounds having molecular weights of more than 50 and less than about 2,500 Dalton, which are more readily absorbed (e.g., after oral administration), have fewer potential antigenic determinants, or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules. Alternatively, candidate agents also include biomolecules such as, but not limited to: nucleic acid polymers, amino acid polymers, saccharides, fatty acids, steroids, purines, pyrimidines, derivatives, structural analogues or combinations thereof. Typically, candidate agents comprise functional groups necessary for structural interaction with polynucleotides or polypeptides, particularly hydrogen bonding, and typically include at least an amine, carbonyl, hydroxyl or carboxyl group, preferably at least two of the functional chemical groups. The candidate agent often comprises cyclical carbon or heterocyclic structures or aromatic or polyaromatic structures substituted with one or more of the above functional groups.

Alternatively, candidate agents can be selected from known pharmacologically active compounds and chemical analogues thereof or from libraries of natural compounds in the form of bacterial, fungal, plant and animal extracts. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc to produce structural analogues.

Screening for modulatory agents according to the invention can be achieved by any suitable method. For example, the method may include contacting a cell expressing a polynucleotide corresponding to an E2F gene or to a gene belonging to the same regulatory pathway as an E2F gene, with an agent suspected of having the modulatory activity and screening for the modulation of the level or functional activity of a protein encoded by the polynucleotide, or the modulation of the level of a transcript encoded by the polynucleotide, or the modulation of the activity or expression of a downstream cellular target of the protein or of the transcript (hereafter referred to as target molecules). Detecting such modulation can be achieved utilising techniques including, but not restricted to, ELISA, cell-based ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which a target molecule of interest is regulated or expressed may be naturally occurring in the cell which is the subject of testing or it may have been introduced into the host cell for the purpose of testing. Further, the naturally-occurring or introduced polynucleotide may be constitutively expressed—thereby providing a model useful in screening for agents which down-regulate expression of an encoded product of the sequence wherein the down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for a target protein or it may comprise a portion of that coding sequence (e.g., an E2F domain selected from the cyclin-binding domain, the DNA-binding domain, the transcription factor E2F/dimerisation partner (TDP) domain, the leucine zipper domain, the heterodimerisation domain, the trans-activation domain and the pocket protein binding domain) or a portion that regulates expression of a product encoded by the polynucleotide (e.g., a promoter). For example, the promoter that is naturally associated with the polynucleotide may be introduced into the cell that is the subject of testing. In this regard, where only the promoter is utilized, detecting modulation of the promoter activity can be achieved, for example, by operably linking the promoter to a suitable reporter polynucleotide including, but not restricted to, green fluorescent protein (GFP), luciferase, β-galactosidase and catecholamine acetyl transferase (CAT). Modulation of expression may be determined by measuring the activity associated with the reporter polynucleotide.

In another example, the subject of detection could be a downstream regulatory target of the target molecule, rather than the target molecule itself or the reporter molecule operably linked to a promoter of a gene encoding a product the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression or activity of a target molecule of the invention.

In a series of embodiments, the present invention provides assays for identifying small molecules or other compounds (i.e., modulatory agents) which are capable of inducing or inhibiting the level and/or functional activity of target molecules according to the invention. The assays may be performed in vitro using non-transformed cells, immortalized cell lines, or recombinant cell lines. In addition, the assays may detect the presence of increased or decreased expression of genes or production of proteins on the basis of increased or decreased mRNA expression (e.g., using nucleic acid probes), increased or decreased levels of protein products (e.g., using antigen binding molecules), or increased or decreased levels of expression of a reporter gene (e.g., GFP, β-galactosidase or luciferase) operably linked to a target molecule-related gene regulatory region in a recombinant construct.

Thus, for example, one may culture cells which produce a particular target molecule (e.g., an E2F or an E2F transcript) and add to the culture medium one or more test compounds. The host cells are suitably epithelial cells, especially human epithelial cells (e.g., skin cells). After allowing a sufficient period of time (e.g., 6-72 hours) for the compound to induce or inhibit the level or functional activity of the target molecule, any change in the level from an established baseline may be detected using any of the techniques described above and well known in the art. In particularly preferred embodiments, the cells are epithelial cells, especially squamous epithelial cells. Using suitable nucleic acid probes or antigen-binding molecules, detection of changes in the level and or functional activity of a target molecule, and thus identification of the compound as agonist or antagonist of the target molecule, requires only routine experimentation.

In some embodiments, recombinant assays are employed in which a reporter gene encoding, for example, GFP, β-galactosidase or luciferase is operably linked to the 5′ regulatory regions of a target molecule related gene. Such regulatory regions may be easily isolated and cloned by one of ordinary skill in the art. The reporter gene and regulatory regions are joined in-frame (or in each of the three possible reading frames) so that transcription and translation of the reporter gene may proceed under the control of the regulatory elements of the target molecule related gene. The recombinant construct may then be introduced into any appropriate cell type, especially cell types of mammalian origin, and more especially of human origin. The transformed cells may be grown in culture and, after establishing the baseline level of expression of the reporter gene, test compounds may be added to the medium. The ease of detection of the expression of the reporter gene provides for a rapid, high throughput assay for the identification of agonists or antagonists of the target molecules of the invention.

Compounds identified by this method will have potential utility in modifying the expression of target molecule related genes in vivo. These compounds may be further tested in the animal models to identify those compounds having the most potent in vivo effects. In addition, as described above with respect to small molecules having target polypeptide binding activity, these molecules may serve as “lead compounds” for the further development of pharmaceuticals by, for example, subjecting the compounds to sequential modifications, molecular modelling, and other routine procedures employed in rational drug design.

In other embodiments, methods of identifying agents that inhibit E2F activity are provided in which a purified preparation of an E2F protein is incubated in the presence and absence of a candidate agent under conditions in which the E2F is active, and the level of E2F activity is measured by a suitable assay. For example, an E2F inhibitor can be identified by measuring the ability of a candidate agent to decrease E2F activity in a cell (e.g., an epithelial cell, especially a squamous epithelial cell). In one embodiment of this method, an epithelial cell that is capable of expressing an E2F gene is exposed to, or cultured in the presence and absence of, the candidate agent under conditions in which the E2F is active in the cells, and an activity relating to cell proliferation and/or differentiation is detected. An agent tests positive if it inhibits this activity.

In still other embodiments, random peptide libraries consisting of all possible combinations of amino acids attached to a solid phase support may be used to identify peptides that are able to bind to a target molecule or to a functional domain thereof. Identification of molecules that are able to bind to a target molecule may be accomplished by screening a peptide library with a recombinant soluble target molecule. The target molecule may be purified, recombinantly expressed or synthesized by any suitable technique. Such molecules may be conveniently prepared by a person skilled in the art using standard protocols as for example described in Sambrook, et al., (1989, supra) in particular Sections 16 and 17; Ausubel et al., (“Current Protocols in Molecular Biology”, John Wiley & Sons Inc, 1994-1998), in particular Chapters 10 and 16; and Coligan et al., (“Current Protocols in Immunology”, (John Wiley & Sons, Inc, 1995-1997), in particular Chapters 1, 5 and 6. Alternatively, a target polypeptide according to the invention may be synthesized using solution synthesis or solid phase synthesis as described, for example, in Chapter 9 of Atherton and Shephard (supra) and in Roberge et al (1995, Science 269: 202).

To identify and isolate the peptide/solid phase support that interacts and forms a complex with a target molecule, suitably a target polypeptide, it may be necessary to label or “tag” the target polypeptide. The target polypeptide may be conjugated to any suitable reporter molecule, including enzymes such as alkaline phosphatase and horseradish peroxidase and fluorescent reporter molecules such as fluorescein isothyiocynate (FITC), phycoerythrin (PE) and rhodamine. Conjugation of any given reporter molecule, with target polypeptide, may be performed using techniques that are routine in the art. Alternatively, target polypeptide expression vectors may be engineered to express a chimeric target polypeptide containing an epitope for which a commercially available antigen-binding molecule exists. The epitope specific antigen-binding molecule may be tagged using methods well known in the art including labelling with enzymes, fluorescent dyes or colored or magnetic beads.

For example, the “tagged” target polypeptide conjugate is incubated with the random peptide library for 30 minutes to one hour at 22° C. to allow complex formation between target polypeptide and peptide species within the library. The library is then washed to remove any unbound target polypeptide. If the target polypeptide has been conjugated to alkaline phosphatase or horseradish peroxidase the whole library is poured into a petri dish containing a substrate for either alkaline phosphatase or peroxidase, for example, 5-bromo-4-chloro-3-indoyl phosphate (BCIP) or 3,3′,4,4″-diamnobenzidine (DAB), respectively. After incubating for several minutes, the peptide/solid phase-target polypeptide complex changes color, and can be easily identified and isolated physically under a dissecting microscope with a micromanipulator. If a fluorescently tagged target polypeptide has been used, complexes may be isolated by fluorescent activated sorting. If a chimeric target polypeptide having a heterologous epitope has been used, detection of the peptide/target polypeptide complex may be accomplished by using a labelled epitope specific antigen-binding molecule. Once isolated, the identity of the peptide attached to the solid phase support may be determined by peptide sequencing.

4. Codon Optimisation of E2F Pathway-modulating Polynucleotides

The codon composition of polynucleotides that code for E2F pathway polypeptide modulators (e.g., anti-E2F antigen-binding molecules and E2F d/n polypeptides) may be altered to enhance the expression of the polypeptide modulators in a selected cell or tissue. Such codon optimisation is predicated on the replacement of existing codons in a parent polynucleotide with synonymous codons that have a higher translational efficiency in a chosen cell or tissue. Any suitable method of replacing synonymous codons for existing codons is contemplated by the present invention. For example, reference may be made to International Application Publication No WO 96/09378 which utilizes substitution of this nature to provide a method of expressing proteins of eukaryotic and viral origin at high levels in in vitro mammalian cell culture systems. Suitably, the codon composition of the polynucleotide is modified to permit selective expression of the polypeptide modulator encoded thereby in a target cell or tissue of choice using methods as set forth in detail in International Application Publication Nos WO 99/02694 and WO 00/42215. In this regard, Frazer et al. were able to show in WO 99/02694 and in copending U.S. application Ser. No. 09/479645 that there are substantial differences in the relative abundance of particular isoaccepting transfer RNAs in different cells or tissues of an organism (e.g., a mammal) and that this plays a pivotal role in protein expression from a coding sequence with a given codon usage or composition. Modification of the codons utilized in transgenes designed to generate translated proteins can lead to much higher and selective expression of particular genes in a cell or tissue of interest. Briefly, the method is based on the observation that translational efficiencies of different codons vary between different cells or tissues. Such differences can be exploited, together with codon composition of a gene, to regulate and direct expression of a protein or a functional fragment or epitope thereof to a particular cell or cell type, including cells in a selected tissue. Codons are selected such that the synonymous codon has a higher translational efficiency in a target cell or tissue than in one or more other cells or tissues.

One or more codons in a gene may be substituted in order to target expression of the gene to particular cells or tissues. It is preferable but not necessary to replace all the existing codons of the parent nucleic acid molecule with synonymous codons having higher translational efficiencies in the target cell or tissue compared to the other cells or tissues. Increased expression can be accomplished even with partial replacement. Suitably, the replacement step affects 5%, 10%, 15%, 20%, 25%, 30%, more preferably 35%, 40%, 50%, 60%, 70% or more of the existing codons of the parent polynucleotide. The difference in level of protein expressed in the desired target cell or tissue from a synthetic polynucleotide, relative to that expressed in the other cells or tissues, depends on the percentage of existing codons replaced by synonymous codons and the difference in translational efficiencies of the synonymous codons in the target cell or tissue, relative to the other cells or tissues.

By optimising codon content, according to the procedures disclosed in WO 99/02694 and in copending U.S. application Ser. No. 09/479645, it has been shown that a protein can be expressed from a synthetic polynucleotide in a target cell or tissue at levels greater than 10,000-fold over those expressed in another cell or tissue. A nucleic acid molecule, which has undergone such codon modification, is referred to herein as “optimized”.

Replacement of one codon for another can be achieved using standard methods known in the art. For example codon modification of a parent polynucleotide can be effected using several known mutagenesis techniques including, for example, oligonucleotide-directed mutagenesis, mutagenesis with degenerate oligonucleotides, and region-specific mutagenesis. Exemplary in vitro mutagenesis techniques are described for example in U.S. Pat. Nos. 4,184,917, 4,321,365 and 4,351,901 or in the relevant sections of Ausubel, et al. (CURRENT PROTOCOLS IN MOLECULAR BIOLOGY, John Wiley & Sons, Inc. 1997) and of Sambrook, et al., (MOLECULAR CLONING. A LABORATORY MANUAL, Cold Spring Harbor Press, 1989). Instead of in vitro mutagenesis, a synthetic polynucleotide with the desired codon composition can be synthesized de novo using readily available machinery as described, for example, in U.S. Pat. No. 4,293,652. However, it should be noted that the present invention is not dependent on, and not directed to, any one particular technique for constructing the synthetic polynucleotide.

Having regard to the above teachings, it is possible to take advantage of different codon translational efficiency profiles displayed between a first cell type and a second cell type to codon modify an polypeptide modulator-encoding polynucleotide, as broadly described above, so that more polypeptide modulator is expressed from the modified polynucleotide in a first cell type than in a second cell type. Accordingly, in certain embodiments in which an E2F pathway polypeptide modulator is used to treat a skin cancer or tumor of epithelial origin, a polynucleotide that codes for the modulator is modified with codons that have a higher translational efficiency in the skin cancer or tumor cells than in corresponding normal epithelial cells.

5. Synthetic Constructs

The invention further contemplates a synthetic construct (or expression vector), comprising a polynucleotide (e.g., encoding an E2F pathway-modulating polypeptide such as an E2F d/n polypeptide or comprising an E2F pathway-modulating nucleic acid molecule such as an E2F-specific antisense, ribozyme or RNAi molecule) of the invention, which is operably linked to a regulatory polynucleotide. The regulatory polynucleotide suitably comprises transcriptional and/or translational control sequences, which will be compatible for expression in the cell or tissue type of interest. Typically, the transcriptional and translational regulatory control sequences include, but are not limited to, a promoter sequence, a 5′ non-coding region, a cis-regulatory region such as a functional binding site for transcriptional regulatory protein or translational regulatory protein, an upstream open reading frame, ribosomal-binding sequences, transcriptional start site, translational start site, and/or nucleotide sequence which encodes a leader sequence, termination codon, translational stop site and a 3′ non-translated region. Constitutive or inducible promoters as known in the art are contemplated by the invention. The promoters may be either naturally occurring promoters, or hybrid promoters that combine elements of more than one promoter. Promoter sequences contemplated by the present invention may be native to the organism of interest or may be derived from an alternative source, where the region is functional in the chosen organism. The choice of promoter will differ depending on the intended host. For example, promoters which could be used for expression in mammalian cells generally include the metallothionein promoter, which can be induced in response to heavy metals such as cadmium, the β-actin promoter as well as viral promoters such as the SV40 large T antigen promoter, human cytomegalovirus (CMV) immediate early (IE) promoter, rous sarcoma virus LTR promoter, adenovirus promoter, or a HPV promoter, particularly the HPV upstream regulatory region (URR) may also be used. All these promoters are well described and readily available in the art. Alternatively, the promoter may be lineage specific and, in this regard, epithelial-specific promoters are particularly desirable such as, but not limited to, promoters of the following genes transglutaminase type 1, involucrin, loricrin, SPR genes and filagrin as well as those of keratin genes (e.g., K10, K14, K5, K1).

The synthetic construct of the present invention may also comprise a 3′ non-translated sequence. A 3′ non-translated sequence refers to that portion of a gene comprising a DNA segment that contains a polyadenylation signal and any other regulatory signals capable of effecting mRNA processing or gene expression. The polyadenylation signal is characterized by effecting the addition of polyadenylic acid tracts to the 3′ end of the mRNA precursor. Polyadenylation signals are commonly recognized by the presence of homology to the canonical form 5′ AATAAA-3′ although variations are not uncommon. The 3′ non-translated regulatory DNA sequence preferably includes from about 50 to 1,000 nucleotide base pairs and may contain transcriptional and translational termination sequences in addition to a polyadenylation signal and any other regulatory signals capable of effecting mRNA processing or gene expression.

In some embodiments, the synthetic construct further contains a screenable marker gene to permit identification of cells containing the synthetic construct. Screenable genes (e.g., lacZ, gfp, etc) are well known in the art and will be compatible for expression in a particular cell or tissue type.

It will be understood, however, that expression of protein-encoding polynucleotides in heterologous systems is now well known, and the present invention is not directed to or dependent on any particular vector, transcriptional control sequence or technique for its production. Rather, synthetic polynucleotides prepared according to the methods as set forth herein may be introduced into selected cells or tissues or into a precursors or progenitors thereof in any suitable manner in conjunction with any suitable synthetic construct or vector, and the synthetic polynucleotides may be expressed with known promoters in any conventional manner

The synthetic constructs can be introduced into suitable host cells for expression using any of a number of non-viral or viral gene delivery vectors. For example, retroviruses (in particular, lentiviral vectors) provide a convenient platform for gene delivery systems. A coding sequence of interest (for example, a sequence useful for gene therapy applications) can be inserted into a gene delivery vector and packaged in retroviral particles using techniques known in the art. Recombinant virus can then be isolated and delivered to cells of the subject either in vivo or ex vivo.

In one illustrative embodiment, retroviruses provide a convenient and effective platform for gene delivery systems. A selected nucleotide sequence encoding an E2F pathway-modulating polypeptide of the present invention can be inserted into a vector and packaged in retroviral particles using techniques known in the art. The recombinant virus can then be isolated and delivered to a subject. Several illustrative retroviral systems have been described examples of which include: U.S. Pat. No. 5,219,740; Miller and Rosman, 1989, Bio Techniques 7: 980-990; Miller, A. D., 1990, Human Gene Therapy 1: 5-14; Scarpa et al., 1991, Virology 180: 849-852; Burns et al., 1993, Proc. Natl. Acad. Sci. USA 90: 8033-8037; and Boris-Lawrie and Temin, 1993, Cur. Opin. Genet. Develop. 3: 102-109).

In addition, several illustrative adenovirus-based systems have also been described. Unlike retroviruses which integrate into the host genome, adenoviruses persist extrachromosomally thus minimising the risks associated with insertional mutagenesis (see, e.g., Haj-Ahmad and Graham, 1986, J. Virol. 57: 267-274; Bett et al., 1993, J. Virol. 67: 5911-5921; Mittereder et al., 1994, Human Gene Therapy 5: 717-729; Seth et al., 1994, J. Virol. 68: 933-940, ; Barr et al., 1994, Gene Therapy 1: 51-58; Berkner, K. L., 1988, Bio Techniques 6: 616-629; and Rich et al., 1993, Human Gene Therapy 4: 461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al., 1988, Molec. Cell. Biol. 8: 3988-3996; Vincent et al., 1990, Vaccines 90, Cold Spring Harbor Laboratory Press; Carter, B. J., 1992, Current Opinion in Biotechnology 3: 533-539; Muzyczka, N., 1992, Current Topics in Microbiol. and Immunol. 158: 97-129; Kotin, R. M., 1994, Human Gene Therapy 5: 793-801; Shelling and Smith, 1994, Gene Therapy 1: 165-169; and Zhou et al., 1994, J. Exp. Med. 179:1867-1875.

Additional viral vectors useful for delivering the polynucleotides encoding the E2F pathway-modulating polypeptides of the present invention by gene transfer include those derived from the pox family of viruses, such as vaccinia virus and avian poxvirus. By way of example, vaccinia virus recombinants expressing an E2F pathway-modulating polypeptide of the invention can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK⁽⁻⁾ recombinant can be selected by culturing the cells in the presence of 5-BrdU and picking viral plaques resistant thereto.

Alternatively, avipoxviruses, such as the fowlpox and canarypox viruses, can also be used to deliver the coding sequences of interest. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al., J. Biol. Chem. 268:6866-69, 1993; and Wagner et al., Proc. Natl. Acad. Sci. USA 89:6099-6103, 1992, can also be used for gene delivery under the invention.

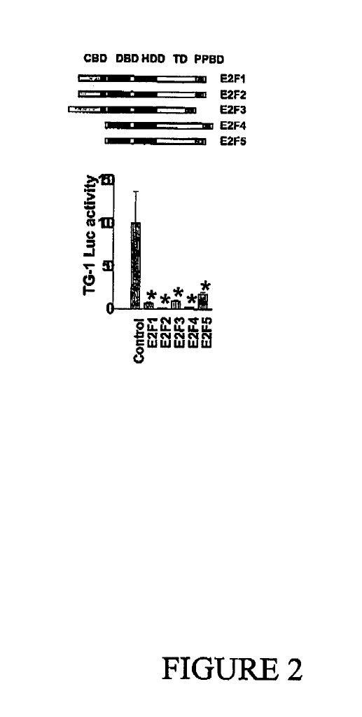

In other illustrative embodiments, lentiviral vectors are employed to deliver the E2F pathway-modulating polypeptide-encoding polynucleotides into selected cells or tissues. Typically, these vectors comprise a 5′ lentiviral LTR, a tRNA binding site, a packaging signal, a promoter operably linked to one or more genes of interest, an origin of second strand DNA synthesis and a 3′ lentiviral LTR, wherein the lentiviral vector contains a nuclear transport element. The nuclear transport element may be located either upstream (5′) or downstream (3′) of a coding sequence of interest (for example, a synthetic Gag or Env expression cassette of the present invention). A wide variety of lentiviruses may be utilized within the context of the present invention, including for example, lentiviruses selected from the group consisting of HIV, HIV-1, HIV-2, FIV, BIV, EIAV, MVV, CAEV, and SIV. Illustrative examples of lentiviral vectors are described in PCT Publication Nos. WO 00/66759, WO 00/00600, WO 99/24465, WO 98/51810, WO 99/51754, WO 99/31251, WO 99/30742, and WO 99/15641. Desirably, a third generation SIN lentivirus is used. Commercial suppliers of third generation SIN (self-inactivating) lentiviruses include Invitrogen (ViraPower Lentiviral Expression System). Detailed methods for construction, transfection, harvesting, and use of lentiviral vectors are given, for example, in the Invitrogen technical manual “ViraPower Lentiviral Expression System version B 050102 25-0501”, available at http://www.invitrogen.com/Content/Tech-Online/molecular_biology/manuals_p-ps/virapower_lentiviral_system_man.pdf. Lentiviral vectors have emerged as an efficient method for gene transfer. Improvements in biosafety characteristics have made these vectors suitable for use at biosafety level 2 (BL2). A number of safety features are incorporated into third generation SIN (self-inactivating) vectors. Deletion of the viral 3′ LTR U3 region results in a provirus that is unable to transcribe a full length viral RNA. In addition, a number of essential genes are provided in trans, yielding a viral stock that is capable of but a single round of infection and integration. Lentiviral vectors have several advantages, including: 1) pseudotyping of the vector using amphotropic envelope proteins allows them to infect virtually any cell type; 2) gene delivery to quiescent, post mitotic, differentiated cells, including neurones, has been demonstrated; 3) their low cellular toxicity is unique among transgene delivery systems; 4) viral integration into the genome permits long term transgene expression; 5) their packaging capacity (6-14 kb) is much larger than other retroviral, or adeno-associated viral vectors. In a recent demonstration of the capabilities of this system, lentiviral vectors expressing GFP were used to infect murine stem cells resulting in live progeny, germline transmission, and promoter-, and tissue-specific expression of the reporter (Ailles, L. E. and Naldini, L., HIV-i-Derived Lentiviral Vectors. In: Trono,D. (Ed.), Lentiviral Vectors, Springer-Verlag, Berlin, Heidelberg, New York, 2002, pp. 31-52). An example of the current generation vectors is outlined in FIG. 2 of a review by Lois et al. (Lois, C., Hong, E. J., Pease, S., Brown, E. J., and Baltimore, D., Germline transmission and tissue-specific expression of transgenes delivered by lentiviral vectors, Science, 295 (2002) 868-872).

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronisation with the host cell cycle. The manner in which the expression construct is delivered to a cell and where in the cell the polynucleotide remains is dependent on the type of expression construct employed.

In other embodiments, a polynucleotide is administered/delivered as “naked” DNA, for example as described in Ulmer et al., Science 259:174549, 1993 and reviewed by Cohen, Science 259:1691-92, 1993. The uptake of naked DNA may be increased by coating the DNA onto biodegradable beads, which are efficiently transported into the cells.

In still other embodiments, a composition of the present invention can be delivered via a particle bombardment approach, many of which have been described. In one illustrative example, gas-driven particle acceleration can be achieved with devices such as those manufactured by Powdeiject Pharmaceuticals PLC (Oxford, UK) and Powdeiject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

6. Therapeutic and Prophylactic Uses

In accordance with the present invention, E2F pathway-antagonising agents are useful as actives for the treatment or prophylaxis of a skin cancer or tumor of epithelial origin. In some embodiments, these agents are separately, simultaneously or sequentially administerable with an agent that stimulates the differentiation of epithelial cells. Typically, these agents are administered to a patient in pharmaceutical compositions where they are mixed with a suitable pharmaceutically acceptable carrier.

The E2F pathway-antagonising agents of the present invention may be conjugated with biological targeting agents which enable their activity to be restricted to particular cell types, especially epithelial cell types. Such biological-targeting agents include substances which are immuno-interactive with cell-specific surface antigens. For example, an agent which modulates the activity of an E2F pathway component (e.g., E2F1) may be conjugated with an agent which is immuno-interactive with a surface protein whose expression is upregulated on a skin cancer cell, especially on a squamous cell carcinoma, e.g., EGF receptor. The presence of this immuno-interactive conjugate confers skin cancer-specificity to the effects of the E2F pathway component-modulating agent. The E2F pathway component-modulating agent can be selected from a compound as broadly described in Section 2 or as identified in Section 3.

Depending on the specific conditions being treated, the drugs may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the drugs of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

The drugs can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of drug administered to a patient should be sufficient to effect a beneficial response in the patient over time such as an enhancement or reduction in adipogenesis. The quantity of the drug(s) to be administered may depend on the subject to be treated inclusive of the age, sex, weight and general health condition thereof. In this regard, precise amounts of the drug(s) for administration will depend on the judgement of the practitioner. In determining the effective amount of the drug to be administered in the modulation of adipogenesis, the physician may evaluate tissue levels of components of the E2F pathway, and degree of cell proliferation and/or differentiation. In any event, those of skill in the art may readily determine suitable dosages of the drugs of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatin, gum tragacanth, methyl cellulose, hydroxypropylmethyl-cellulose, sodium carboxymethylcellulose, or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more drugs as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments may be added to the tablets or dragee coatings for identification or to characterize different combinations of active compound doses.

Pharmaceutical which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dosage forms of the drugs of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes or microspheres.

The drugs of the invention may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

One may also administer the compound(s) in a local rather than systemic manner, for example, via injection of the compound(s) directly into a tissue, which is suitably dermal or epidermal tissue, often in a depot or sustained release formulation.

Furthermore, one may administer the compound(s) in a targeted drug delivery system, for example, in a liposome coated with tissue-specific antibody. The liposomes will be targeted to and taken up selectively by the tissue.

In cases of local administration or selective uptake, the effective local concentration of the agent may not be related to plasma concentration.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of a test agent, which achieves a half-maximal inhibition or enhancement in activity of an E2F pathway component polypeptide). Such information can be used to more accurately determine useful doses in humans.

Toxicity and therapeutic efficacy of such drugs can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See for example Fingl et al., 1975, in “The Pharmacological Basis of Therapeutics”, Ch. 1 p1).

Dosage amount and interval may be adjusted individually to provide plasma levels of the active agent which are sufficient to maintain FGF or FGFR-inhibitory or enhancement effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mglkg/day. Stated in terms of patient body surface areas, usual dosages range from 0.5-1200 mg/m²/day, commonly from 0.5-150 mg/m²/day, typically from 5-100 mg/m²/day.

Thus, in accordance with the present invention, an E2F pathway antagonising agent can be used to antagonize the E2F pathway in epithelial cells to thereby inhibit proliferation of those cells, and to induce or potentiate their differentiation. In some embodiments, a differentiation-stimulating agent is used in concert with the E2F pathway antagonising agent to permit the differentiation of the epithelial cells. In related embodiments, this antagonism is used in skin cancers therapy to reverse or reduce the malignant phenotype of the skin cancer cells. The E2F pathway antagonising agent and the differentiation-stimulating agent may be provided in effective amounts to inhibit proliferation of the cancer cells and induce their differentiation. This process may involve contacting the cells with the E2F pathway antagonising agent separately, simultaneously or sequentially with the differentiation-stimulating agent. In some embodiments, this may be achieved by contacting the cells with a single composition or pharmacological formulation that includes both agents, or by contacting the cell with two distinct compositions or formulations at the same time, wherein one composition includes the E2F pathway antagonising agent and the differentiation-stimulating agent. In other embodiments, the treatment with the E2F pathway antagonising agent may precede or follow the treatment with the differentiation-stimulating agent by intervals ranging from minutes to days. In embodiments where the differentiation-stimulating agent is applied separately to the cells, one would generally ensure that a significant period of time did not expire between the time of each delivery, such that differentiation-stimulating agent would still be able to exert an advantageously combined effect on the cells. In such instances, it is contemplated that one would contact the cell with both modalities within about 1-12 hours of each other and, more suitably, within about 2-6 hours of each other. In some situations, it may be desirable to extend the time period for treatment significantly, however, where several hours (2, 3, 4, 5, 6 or 7) to several days (1, 2, 3, 4, 5, 6, 7 or 8) lapse between the respective administrations.

It also is conceivable that more than one administration of either E2F pathway antagonising agent or differentiation-stimulating agent will be desired. Various combinations may be employed, where the E2F pathway antagonising agent is “A” and the differentiation-stimulating agent is “B”, as exemplified below:
A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B

Other combinations are contemplated. Again, both agents are delivered to cells in a combined amount effective to inhibit proliferation of the cancer cells and to induce their differentiation.

The present invention also contemplates the use of synthetic constructs in methods for inhibiting proliferation of epithelial cells, especially skin cancer cells and epithelial cells of tumor origin, and for inducing or potentiating the differentiation of those cells. a method of gene therapy of a mammal, as described in Section 5, supra. Administration of such constructs to a mammal, especially a human, may include delivery via direct oral intake, systemic injection, or delivery to selected tissue(s) or cells. Delivery of the constructs to cells or tissues of the mammal may be facilitated by microprojectile bombardment, liposome mediated transfection (e.g., lipofectin or lipofectamine), electroporation, calcium phosphate or DEAE-dextran-mediated transfection, for example. A discussion of suitable delivery methods may be found in Chapter 9 of Ausubel et al., (1994-1998, supra).

The step of introducing the expression vector into the selected target cell or tissue will differ depending on the intended use and species, and can involve one or more of non-viral and viral vectors, cationic liposomes, retroviruses, and adenoviruses such as, for example, described in Mulligan, R. C., (1993). Such methods can include, for example:
A. Local application of the expression vector by injection (Wolff et al., 1990), surgical implantation, instillation or any other means. This method can also be used in combination with local application by injection, surgical implantation, instillation or any other means, of cells responsive to the protein encoded by the expression vector so as to increase the effectiveness of that treatment. This method can also be used in combination with local application by injection, surgical implantation, instillation or any other means, of another factor or factors required for the activity of the protein.
B. General systemic delivery by injection of DNA, (Calabretta et al., 1993), or RNA, alone or in combination with liposomes (Zhu et al., 1993), viral capsids or nanoparticles (Bertling et al., 1991) or any other mediator of delivery. Improved targeting might be achieved by linking the polynucleotide/expression vector to a targeting molecule (the so-called “magic bullet” approach employing, for example, an antigen-binding molecule), or by local application by injection, surgical implantation or any other means, of another factor or factors required for the activity of the protein encoded by the expression vector, or of cells responsive to the protein. For example, in the case of a liposome containing antisense E2F polynucleotides, the liposome may be targeted to skin cancer cells, especially to squamous cell carcinoma cells, by the incorporation of immuno-interactive agents into the liposome coat which are specific for skin cancer-specific cell surface antigens. An example of a skin cancer-specific cell surface antigen is the EGF receptor.
C. Injection or implantation or delivery by any means, of cells that have been modified ex vivo by transfection (for example, in the presence of calcium phosphate: Chen et al., 1987, or of cationic lipids and polyamines: Rose et al., 1991), infection, injection, electroporation (Shigekawa et al., 1988) or any other way so as to increase the expression of the polynucleotide in those cells. The modification can be mediated by plasmid, bacteriophage, cosmid, viral (such as adenoviral or retroviral; Mulligan, 1993; Miller, 1992; Salmons et al., 1993) or other vectors, or other agents of modification such as liposomes (Zhu et al., 1993), viral capsids or nanoparticles (Bertling et al., 1991), or any other mediator of modification. The use of cells as a delivery vehicle for genes or gene products has been described by Barr et al., 1991 and by Dhawan et al., 1991. Treated cells can be delivered in combination with any nutrient, growth factor, matrix or other agent that will promote their survival in the treated subject.
   7. Methods of Detecting Aberrant E2F Pathway Marker Gene Expression

The present invention discloses the discovery that overexpression of E2F in keratinocyte results in deregulated proliferation and repression of terminal differentiation of these cells. It is proposed, therefore, that aberrant expression of an E2F pathway maker gene as herein defined is associated with the presence, stage, degree or risk of development of a skin cancer or tumor of epithelial origin.

Accordingly, in certain embodiments, the invention features a method for diagnosing the presence, absence, degree or stage of a skin cancer or tumor of epithelial origin in a subject by detecting aberrant expression of an E2F pathway marker gene in a biological sample obtained from the subject. In order to make such diagnoses, it will be desirable to qualitatively or quantitatively determine the levels of E2F pathway marker gene transcripts or the level or functional activity of E2F pathway marker polypeptides. In some embodiments, the presence, degree, stage or risk of development of E2F pathway is diagnosed when an E2F pathway marker gene product is expressed at a detectably higher level in the biological sample as compared to the level at which that gene is expressed in a reference sample obtained from normal subjects or from subjects lacking the skin cancer or tumor. In other embodiments, the presence, degree, stage or risk of development of skin cancer or tumor of epithelial origin is diagnosed when an E2F pathway marker gene product is expressed at a detectably lower level in the biological sample as compared to the level at which that gene is expressed in a reference sample obtained from normal subjects or from subjects lacking skin cancer or tumor. Generally, such diagnoses are made when the level or functional activity of an E2F pathway marker gene product in the biological sample varies from the level or functional activity of a corresponding E2F pathway marker gene product in the reference sample by at least 10%, 20%, 30%, 40%, 50%, 60%, 70%, 80%, 90%, 92%, 94%, 96%, 97%, 98% or 99%, or even by at least about 99.5%, 99.9%, 99.95%, 99.99%, 99.995% or 99.999%, or even by at least about 100%, 200%, 300%, 400%, 500%, 600%, 700%, 800%, 900%, or 1000%.

The corresponding gene product is generally selected from the same gene product that is present in the biological sample, a gene product expressed from a variant gene (e.g., an homologous or orthologous gene) including an allelic variant, or a splice variant or protein product thereof. In some embodiments, the method comprises measuring the level or functional activity of individual expression products of at least about 2, 3, 4 or 5 E2F pathway marker genes.

Generally, the biological sample contains a tissue biopsy, which suitably contains an epithelial cell, especially a squamous epithelial cell, such as from epidermis, oral mucosa, oesophageal, vaginal, tracheal or corneal epithelia

7.1 Nucleic Acid-based Diagnostics

Nucleic acid used in polynucleotide-based assays can be isolated from cells contained in the biological sample, according to standard methodologies (Sambrook, et al., 1989, supra; and Ausubel et al., 1994, supra). The nucleic acid is typically fractionated (e.g., poly A⁺RNA) or whole cell RNA. Where RNA is used as the subject of detection, it may be desired to convert the RNA to a complementary DNA. In some embodiments, the expression of a target nucleic acid is measured in a sample directly, for example, by conventional Southern blotting, Northern blotting to quantitate the transcription of mRNA (Thomas, 1980, Proc. Natl. Acad. Sci. USA, 77:5201-5205), dot blotting (DNA analysis), or in situ hybridisation, using an appropriately labelled probe. Alternatively, antibodies may be employed that can recognize specific duplexes, including DNA duplexes, RNA duplexes, and DNA-RNA hybrid duplexes or DNA-protein duplexes. The antibodies in turn may be labelled and the assay may be carried out where the duplex is bound to a surface, so that upon the formation of duplex on the surface, the presence of antibody bound to the duplex can be detected.

In some embodiments, the nucleic acid is amplified by a template-dependent nucleic acid amplification technique. A number of template dependent processes are available to amplify the E2F pathway marker sequences present in a given template sample. An exemplary nucleic acid amplification technique is the polymerase chain reaction (referred to as PCR) which is described in detail in U.S. Pat. Nos. 4,683,195, 4,683,202 and 4,800,159, Ausubel et al. (supra), and in Innis et al., (“PCR Protocols”, Academic Press, Inc., San Diego Calif., 1990). Briefly, in PCR, two primer sequences are prepared that are complementary to regions on opposite complementary strands of the marker sequence. An excess of deoxynucleoside triphosphates are added to a reaction mixture along with a DNA polymerase, e.g., Taq polymerase. If a cognate E2F pathway marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated. A reverse transcriptase PCR amplification procedure may be performed in order to quantify the amount of mRNA amplified. Methods of reverse transcribing RNA into cDNA are well known and described in Sambrook et al., 1989, supra. Alternative methods for reverse transcription utilize thermostable, RNA-dependent DNA polymerases. These methods are described in WO 90/07641. Polymerase chain reaction methodologies are well known in the art.

In certain advantageous embodiments, the template-dependent amplification involves the quantification of transcripts in real-time. For example, RNA or DNA may be quantified using the Real-Time PCR technique (Higuchi, 1992, et al., Biotechnology 10: 413-417). By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundance of the specific mRNA from which the target sequence was derived can be determined for the respective tissues or cells. This direct proportionality between the concentration of the PCR products and the relative mRNA abundance is only true in the linear range of the PCR reaction. The final concentration of the target DNA in the plateau portion of the curve is determined by the availability of reagents in the reaction mix and is independent of the original concentration of target DNA.

Another method for amplification is the ligase chain reaction (“LCR”), disclosed in EPO No. 320 308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

QP Replicase, described in PCT Application No. PCT/US87/00880, may also be used. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′α-thio-triphosphates in one strand of a restriction site may also be useful in the amplification of nucleic acids in the present invention, Walker et al., (1992, Proc. Natl. Acad. Sci. US.A 89: 392-396).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridisation, the reaction is treated with RNase H, and the products of the probe identified as distinctive products that are released after digestion. The original template is annealed to another cycling probe and the reaction is repeated.

Still another amplification method described in GB Application No. 2 202 328, and in PCT Application No. PCT/US89/01025, may be used. In the former application, “modified” primers are used in a PCR-like, template- and enzyme-dependent synthesis. The primers may be modified by labelling with a capture moiety (e.g., biotin) and/or a detector moiety (e.g., enzyme). In the latter application, an excess of labelled probes are added to a sample. In the presence of the target sequence, the probe binds and is cleaved catalytically. After cleavage, the target sequence is released intact to be bound by excess probe. Cleavage of the labelled probe signals the presence of the target sequence.

Other nucleic acid amplification procedures include transcription-based amplification systems (TAS), including nucleic acid sequence based amplification (NASBA) and 3SR (Kwoh et al., 1989, Proc. Natl. Acad. Sci. U.S.A., 86: 1173; Gingeras et al., PCT Application WO 88/10315). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerisation, DNA/RNA hybrids are digested with RNase H while double stranded DNA molecules are heat denatured again. In either case the single stranded DNA is made fully double stranded by addition of second target specific primer, followed by polymerisation. The double-stranded DNA molecules are then multiply transcribed by an RNA polymerase such as T7 or SP6. In an isothermal cyclic reaction, the RNAs are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 disclose a nucleic acid amplification process involving cyclically synthesising single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al. in PCT Application WO 89/06700 disclose a nucleic acid sequence amplification scheme based on the hybridisation of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, M. A., In: “PCR Protocols: A Guide to Methods and Applications”, Academic Press, N.Y., 1990; Ohara et al., 1989, Proc. Natl Acad. Sci. U.S.A., 86: 5673-567).

Methods based on ligation of two (or more) oligonucleotides in the presence of nucleic acid having the sequence of the resulting “di-oligonucleotide”, thereby amplifying the di-oligonucleotide, may also be used for amplifying target nucleic acid sequences. Wu et al., (1989, Genomics 4: 560).

Depending on the format, the E2F pathway marker nucleic acid of interest is identified in the sample directly using a template-dependent amplification as described, for example, above, or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994, J Macromol. Sci. Pure, Appl. Chem., A31(1): 1355-1376).

In some embodiments, amplification products or “amplicons” are visualized in order to confirm amplification of the E2F pathway marker sequences. One typical visualisation method involves staining of a gel with ethidium bromide and visualisation under UV light. Alternatively, if the amplification products are integrally labelled with radio- or fluorometrically-labelled nucleotides, the amplification products can then be exposed to x-ray film or visualized under the appropriate stimulating spectra, following separation. In some embodiments, visualisation is achieved indirectly. Following separation of amplification products, a labelled nucleic acid probe is brought into contact with the amplified E2F pathway marker sequence. The probe is suitably conjugated to a chromophore but may be radiolabelled. Alternatively, the probe is conjugated to a binding partner, such as an antigen-binding molecule, or biotin, and the other member of the binding pair carries a detectable moiety or reporter molecule. The techniques involved are well known to those of skill in the art and can be found in many standard texts on molecular protocols (e.g., see Sambrook et al., 1989, supra and Ausubel et al. 1994, supra). For example, chromophore or radiolabel probes or primers identify the target during or following amplification.

In certain embodiments, target nucleic acids are quantified using blotting techniques, which are well known to those of skill in the art. Southern blotting involves the use of DNA as a target, whereas Northern blotting involves the use of RNA as a target. Each provide different types of information, although cDNA blotting is analogous, in many aspects, to blotting or RNA species. Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (usually labelled) under conditions that promote denaturation and rehybridisation. Because the probe is designed to base pair with the target, the probe will bind a portion of the target sequence under renaturing conditions. Unbound probe is then removed, and detection is accomplished as described above.

Following detection/quantification, one may compare the results seen in a given subject with a control reaction or a statistically significant reference group of normal subjects or of subjects lacking E2F pathway. In this way, it is possible to correlate the amount of a E2F pathway marker nucleic acid detected with the progression or severity of the disease.

Also contemplated are genotyping methods and allelic discrimination methods and technologies such as those described by Kristensen et al. (Biotechniques 30(2): 318-322), including the use of single nucleotide polymorphism analysis, high performance liquid chromatography, TaqMan®, liquid chromatography, and mass spectrometry.

Also contemplated are biochip-based technologies such as those described by Hacia et al. (1996, Nature Genetics 14: 441447) and Shoemaker et al. (1996, Nature Genetics 14: 450456). Briefly, these techniques involve quantitative methods for analysing large numbers of genes rapidly and accurately. By tagging genes with oligonucleotides or using fixed probe arrays, one can employ biochip technology to segregate target molecules as high density arrays and screen these molecules on the basis of hybridisation. See also Pease et al. (1994, Proc. Natl. Acad. Sci. U.S.A. 91: 5022-5026); Fodor et al. (1991, Science 251: 767-773). Briefly, nucleic acid probes to E2F pathway marker polynucleotides are made and attached to biochips to be used in screening and diagnostic methods, as outlined herein. The nucleic acid probes attached to the biochip are designed to be substantially complementary to specific expressed E2F pathway marker nucleic acids, i.e., the target sequence (either the target sequence of the sample or to other probe sequences, for example in sandwich assays), such that hybridisation of the target sequence and the probes occurs. This complementarity need not be perfect; there may be any number of base pair mismatches which will interfere with hybridisation between the target sequence and the nucleic acid probes of the present invention. However, if the number of mismatches is so great that no hybridisation can occur under even the least stringent of hybridisation conditions, the sequence is not a complementary target sequence. In certain embodiments, more than one probe per sequence is used, with either overlapping probes or probes to different sections of the target being used. That is, two, three, four or more probes, with three being desirable, are used to build in a redundancy for a particular target. The probes can be overlapping (i.e. have some sequence in common), or separate.

7.2 Protein-based Diagnostics

Consistent with the present invention, the presence of an aberrant concentration of an E2F pathway marker protein is indicative of the presence, degree, stage, or risk of development of a skin caner or tumor of epithelial origin. E2F pathway marker protein levels in biological samples can be assayed using any suitable method known in the art. For example, when an E2F pathway marker protein is an enzyme, the protein can be quantified based upon its catalytic activity or based upon the number of molecules of the protein contained in a sample. Antibody-based techniques may be employed, such as, for example, immunohistological and immunohistochemical methods for measuring the level of a protein of interest in a tissue sample. For example, specific recognition is provided by a primary antibody (polyclonal or monoclonal) and a secondary detection system is used to detect presence (or binding) of the primary antibody. Detectable labels can be conjugated to the secondary antibody, such as a fluorescent label, a radiolabel, or an enzyme (e.g., alkaline phosphatase, horseradish peroxidase) which produces a quantifiable, e.g., colored, product. In another suitable method, the primary antibody itself can be detectably labelled. As a result, immunohistological labelling of a tissue section is provided. In some embodiments, a protein extract is produced from a biological sample (e.g., tissue, cells) for analysis. Such an extract (e.g., a detergent extract) can be subjected to western-blot or dot/slot assay of the level of the protein of interest, using routine immunoblotting methods (Jalkanen et al., 1985, J. Cell. Biol. 101: 976-985; Jalkanen et al., 1987, J. Cell. Biol. 105: 3087-3096).

Other useful antibody-based methods include immunoassays, such as the enzyme-linked immunosorbent assay (ELISA) and the radioimmunoassay (RIA). For example, a protein-specific monoclonal antibody, can be used both as an immunoadsorbent and as an enzyme-labelled probe to detect and quantify an E2F pathway marker protein of interest. The amount of such protein present in a sample can be calculated by reference to the amount present in a standard preparation using a linear regression computer algorithm (see Lacobilli et al., 1988, Breast Cancer Research and Treatment 11: 19-30). In other embodiments, two different monoclonal antibodies to the protein of interest can be employed, one as the immunoadsorbent and the other as an enzyme-labelled probe.

An alternative to an array of capture molecules is one made through ‘molecular imprinting’ technology, in which peptides (e.g., from the C-terminal regions of proteins) are used as templates to generate structurally complementary, sequence-specific cavities in a polymerisable matrix; the cavities can then specifically capture (denatured) proteins which have the appropriate primary amino acid sequence (e.g., available from ProteinPrint™ and Aspira Biosystems).

Exemplary protein capture arrays include arrays comprising spatially addressed antigen-binding molecules, commonly referred to as antibody arrays, which can facilitate extensive parallel analysis of numerous proteins defining a proteome or subproteome. Antibody arrays have been shown to have the required properties of specificity and acceptable background, and some are available commercially (e.g., BD Biosciences, Clontech, BioRad and Sigma). Various methods for the preparation of antibody arrays have been reported (see, e.g., Lopez et al., 2003 J. Chromatogr. B 787:19-27; Cahill, 2000 Trends in Biotechnology 7:47-51; U.S. pat. app. Pub. 2002/0055186; U.S. pat. app. Pub. 2003/0003599; PCT publication WO 03/062444; PCT publication WO 03/077851; PCT publication WO 02/59601; PCT publication WO 02/39120; PCT publication WO 01/79849; PCT publication WO 99/39210). The antigen-binding molecules of such arrays may recognize at least a subset of proteins expressed by a cell or population of cells, illustrative examples of which include growth factor receptors, hormone receptors, neurotransmitter receptors, catecholamine receptors, amino acid derivative receptors, cytokine receptors, extracellular matrix receptors, antibodies, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors, cell-surface antigens, hepatitis C virus (HCV) proteases and HIV proteases.

Particles in suspension can also be used as the basis of arrays, providing they are coded for identification; systems include color coding for microbeads (e.g., available from Luminex, Bio-Rad and Nanomics Biosystems) and semiconductor nanocrystals (e.g., QDots™, available from Quantum Dots), and barcoding for beads (UltraPlex™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual protein-capture agents are typically attached to an individual particle to provide the spatial definition or separation of the array. The particles may then be assayed separately, but in parallel, in a compartmentalized way, for example in the wells of a microtitre plate or in separate test tubes.

In certain embodiments, the techniques used for detection of E2F pathway marker expression products will include internal or external standards to permit quantitative or semi-quantitative determination of those products, to thereby enable a valid comparison of the level or functional activity of these expression products in a biological sample with the corresponding expression products in a reference sample or samples. Such standards can be determined by the skilled practitioner using standard protocols. In specific examples, absolute values for the level or functional activity of individual expression products are determined.

7.3 Kits

All the essential materials and reagents required for detecting and quantifying E2F pathway maker gene expression products may be assembled together in a kit. The kits may also optionally include appropriate reagents for detection of labels, positive and negative controls, washing solutions, blotting membranes, microtitre plates dilution buffers and the like. For example, a nucleic acid-based detection kit may include (i) an E2F pathway marker polynucleotide (which may be used as a positive control), (ii) a primer or probe that specifically hybridizes to an E2F pathway marker polynucleotide. Also included may be enzymes suitable for amplifying nucleic acids including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. Alternatively, a protein-based detection kit may include (i) an E2F pathway marker polypeptide (which may be used as a positive control), (ii) an antigen-binding molecule that is immuno-interactive with an E2F pathway marker polypeptide. The kit can also feature various devices and reagents for performing one of the assays described herein; and/or printed instructions for using the kit to quantify the expression of an E2F pathway marker gene.

In order that the invention may be readily understood and put into practical effect, particular preferred embodiments will now be described by way of the following non-limiting example.

### EXAMPLES

### Example 1

E2Fs 1-5 Can Suppress the Activity of Diferentiation-specific Markers in Normal Human Keratinocytes

In order to determine whether the overexpression of E2F 1 observed in SCCs (2) could affect the ability of the cells to undergo squamous differentiation, the inventor examined the effects of E2F overexpression in normal keratinocytes.

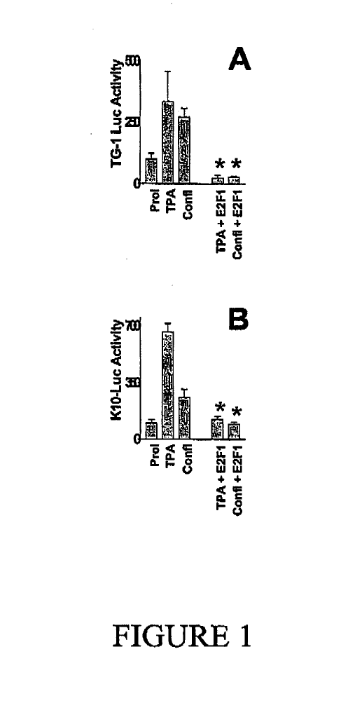

Keratinocyte differentiation is characterized by irreversible growth arrest, the suppression of proliferation-specific markers (1, 49) and the induction of differentiation-specific markers (e.g., transglutaminase-1 (TG-1) or keratin 10 (K10)). The implications of E2F1 overexpression in normal keratinocytes were examined, therefore, by measuring differentiation-specific marker activity in cells induced to differentiate by two independent pathways (confluence or PKC activation; (41, 50, 51)). The results demonstrate that cultured HEKs have increased TG-1 Luc (TG-1 promoter linked to a firefly luciferase gene) and K10-Luc (K10 promoter linked to a firefly luciferase gene) activity when induced to differentiate either by treatment of cells with TPA or by maintaining cells at confluence for 48 hours (FIG. 1). Upon transfection of E2F1 into these differentiated cells, activity of these differentiation-specific markers was significantly suppressed. This observation indicates that E2F1 can suppress differentiation-specific reporters in keratinocytes induced to differentiate by two different stimuli.

In addition, co-transfection of E2Fs 1-5 and the TG-1 Luc reporter into differentiated keratinocytes showed that E2Fs 1-5 were all able to significantly suppress TG-1 Luc activity in confluent/differentiated HEKs (FIG. 2). This indicates that the ability to suppress differentiation markers is shared by E2Fs 1-5. Since E2Fs 4-5 do not contain a cyclin binding domain (19, 22) and are unable to induce apoptosis (34-37, 52-55), these data also suggest that the phenomenon observed is cell-cycle phase-independent, cyclin-binding domain-independent and apoptosis-independent.

Materials and Methods

Cell Culture

Human epidermal keratinocytes (HEKs) were isolated and cultured from neonatal foreskins as described (1). The SCC cell line, KJD-1/SV40, were grown in culture as previously reported (2). Growth arrest and differentiation was induced by maintaining confluent cells in culture over 48 hours or by treatment with the protein kinase C (PKC) activator, 12-Otetradecanoyl-phorbol-13-acetate (TPA, 50 ng/ml for 48 hours). Both these treatment regimes induce robust and reproducible differentiation in HEKs but not in SCC cell lines (2, 41-43).

Transfection of Cells and Reporter Assays

The human cdc2 promoter construct driving expression of a CAT reporter gene (cdc2-CAT) has been previously described (6). The 2.9kb transglutaminase type 1 promoter linked to a firefly luciferase gene (TG-1 Luc), keratin 10 reporter (K10-Luc), β actin-CAT reporter gene and β actin-luciferase reporter gene have also been previously described (2, 6, 41, 44, 45). β actin-CAT or β actin-Luc reporters were used to normalize for transfection efficiency. The CMV-E2F1 construct was a kind gift from Dr Kristian Helin (16). The CMV-E2F 2-5 constructs were generous gifts from Dr David Livingston (46). The mutant E2F1 constructs, 132E2F1 and 409E2F1, were kindly provided by Dr Joseph Nevins (47). The E2F1 dominant-negative (E2Fd/n) construct codes for amino acids 116-235, spanning for the DNA binding domain and heterodimerisation domain (2).

Transient transfections of cells were performed in 10 cm² well, when cells were either 50% or 100% confluent. Reporter activity was assayed 48 hours post-transfection. Transfection protocols for cultured HEKs using Lipofectamine™ (Invitrogen, Australia) and KJD-1/SV40 cells using Effectene (Qiagen, Australia) have been described previously (2). Transfections were performed in triplicate and repeated at least three times.

Chloramphenicol acetyltransferase assays (CAT) were performed using a CAT ELISA kit (Roche, Australia) as per manufacturer's instructions. The luciferase assay protocol has been previously reported (6, 41).

### Example 2

The DNA Binding Domain and Trans-activation Domain of E2F1 is Essential for Suppression of Differentiation-Specific Marker Activity

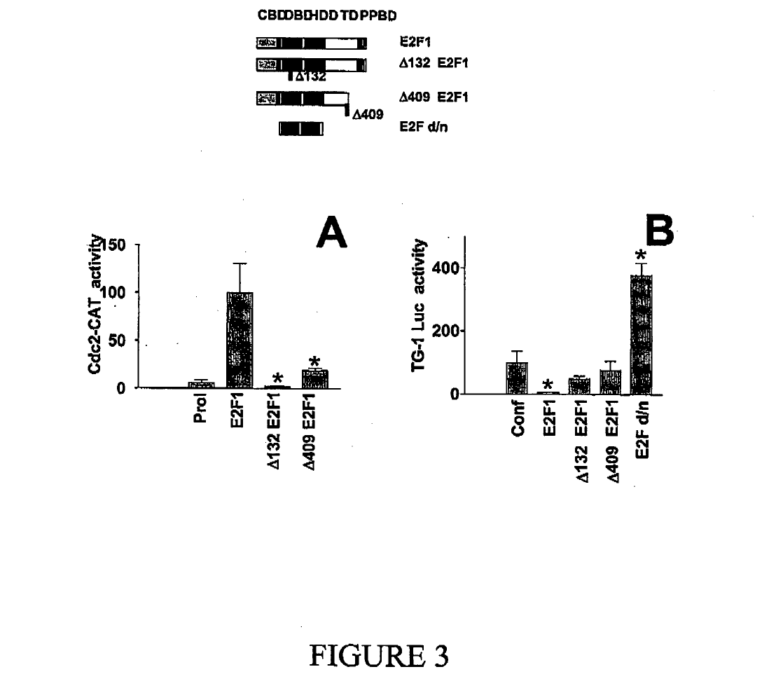

To examine the domain requirement of E2F1 to suppress squamous differentiation, we employed two E2F1 mutants (FIG. 3). The 132E2F1 mutant contains a point mutation in the DNA binding domain which abolishes its activity (47). Similarly, the 409E2F1 mutant possesses a frameshift mutation that eliminates both the trans-activation domain and pocket protein binding domain of E2F1 (47). Both the DNA binding domain mutant (132E2F1) and trans-activation domain mutant (409E2F1) are unable to induce the proliferation-specific and E2F-responsive cdc2-CAT reporter (FIG. 3A). Furthermore, by measuring TG-1 promoter activity, we show that both mutants are unable to suppress TG-1 Luc activity in differentiated keratinocytes (FIG. 3B). These data demonstrate that both the DNA binding domain of E2F1 and the trans-activation domain of E2F1 are important for the suppression of differentiation-specific markers in HEKs. These data also suggest that suppression of differentiation is unlikely to be mediated by “squelching.” It is interesting to note that E2Fd/n was able to superinduce/derepress TG-1 Luc activity to levels above that of differentiated HEKs. Thus, E2F1 overexpression suppresses TG-1 Luc and E2F inhibition induces TG-1 Luc. These data are consistent with a role for E2F as a modulator of squamous differentiation.

### Example 3

E2F is Required for but not Sufficient to Induce Squamous Differentiation

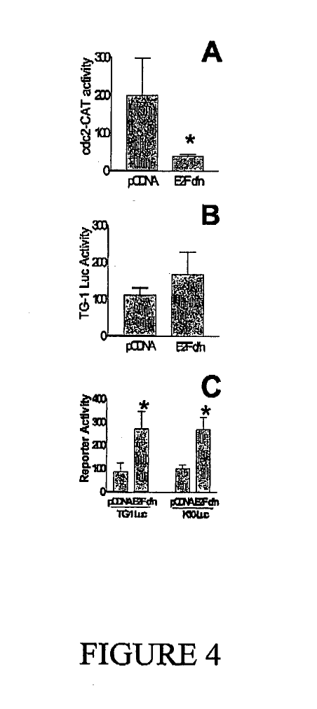

Given that the E2Fd/n could further induce TG-1 Luc activity in confluent cells, the inventor examined the possibility that in proliferative cells the induction of differentiation was actively suppressed by E2F. If this were true, then inhibition of E2F in proliferative cells should suppress proliferation markers and derepress differentiation markers. Indeed, inhibition of E2F causes suppression of the proliferation-specific marker activity, cdc2-CAT (FIG. 4A). However, inhibition of E2F is not sufficient to induce TG-1 Luc activity (FIG. 4B). Paradoxically, inhibition of E2F in confluent/differentiated HEKs superinduces/derepresses both TG-1 Luc and K10-Luc activity (FIG. 4C). These data indicate that (i) E2F suppression is not sufficient to induce differentiation, (ii) E2F inhibits the initiation of squamous differentiation (FIG. 1) and (iii) E2F inhibition is able to derepress/superinduce differentiation markers in differentiated HEKs. For these reasons, the present inventor proposes that E2F acts as a modulator of the differentiation phenotype.

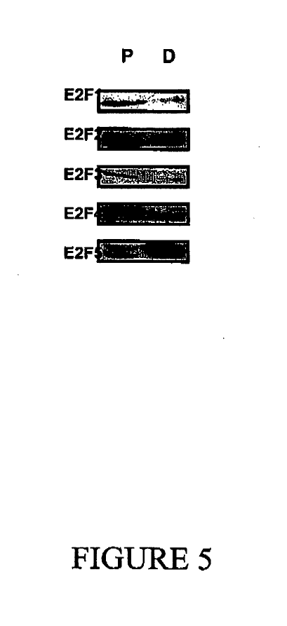

If E2F were to act as a modulator of squamous differentiation in keratinocytes, it would be of interest to determine which E2F isoforms are expressed in differentiated keratinocytes and hence which isoforms potentially contribute to the suppression. Whole cell extracts were blotted and probed with E2F 1-5 antibodies. Both proliferating and confluent HEKs were found to express E2Fs 1-5 protein (FIG. 5). These data indicate that any one of the E2F isoforms could have the potential to modulate squamous differentiation. E2F5 was the only isoform whose expression was increased whilst E2F2 was the only isoform whose expression was decreased in differentiated cells.

Materials and Methods

Protein Isolation and Western Blotting

Protein isolation and western blotting protocols have been previously described (48). All antibodies were purchased from Santa Cruz Biotechnologies (Santa Cruz, USA). Dilutions of rabbit polyclonal antibodies were as follows: E2F1 (sc-193) 1/200, E2F2 (sc-632) 1/1000, E2F3 (sc-878) 1/200, E2F4 (sc-866) 1/1000 and E2F5 (sc-999) 1/1000. All immunodetections were visualized after reaction with 1:3000 HRP-conjugated goat anti-rabbit and incubation with ECL reagent (48).

### Example 4

Inhibition of E2F in the Presence of a Differentiation-inducing Agent Reinstates TG-1 Luc Activity in a Squamous Cell Carcinoma Cell Line

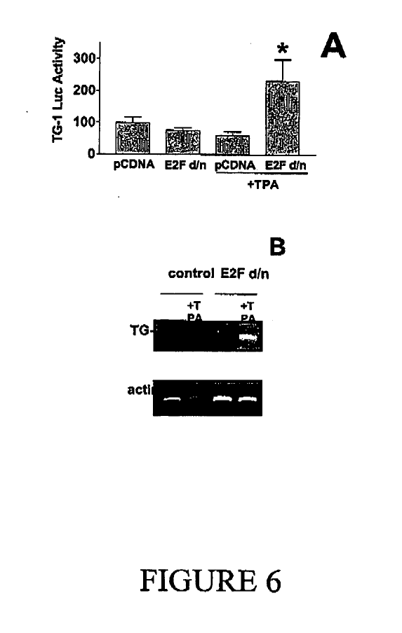

If E2F acts as a modulator of differentiation, this may explain the differentiation-resistance observed in cancer cells in which E2F is overexpressed. The KJD-1/SV40 cells represent a SCC cell line that was produced by transforming normal keratinocytes with the SV40 virus. These cells were used in transfection studies in order to determine whether the inhibition of E2F was able to reinstate TG-i Luc activity. When proliferating KJD-1/SV40 cells were transfected with E2Fd/n, TG-1 Luc activity was not altered (FIG. 6). Similarly, when proliferating KJD-1/SV40 cells were treated with TPA, there was no induction of TG-1 Luc activity. However, treatment of KJD-1/SV40 cells with both a differentiation-inducing agent (TPA) and E2Fd/n resulted in a significant increase in TG-1 Luc activity (FIG. 6).

### Discussion of the Examples

The above experimental data clearly show that the E2F family of molecules are potent and biologically-relevant modulators of squamous differentiation. This extends the known functions of E2F as key regulators of proliferation (56, 57) and apoptosis (56, 58, 59) to include regulation of epithelial cell, especially keratinocyte, terminal differentiation. A role for the E2F family as differentiation modulators is based on the following observations: (i) E2F1 suppresses differentiation-specific markers regardless of stimuli used to induce squamous differentiation, (ii) the ability to suppress squamous differentiation is shared by E2Fs 1-5, (iii) inhibition of E2F is not sufficient to induce keratinocyte differentiation but can superinduce/derepress differentiation markers in differentiated cells, (iv) the superinduction/derepression mediated by an E2F dominant negative polypeptide was observed for both transglutaminase-1 and keratin 10 promoter activity, (v) E2F isoforms are expressed in both proliferating and differentiated keratinocytes, (vi) differentiation-insensitive squamous cell carcinomas (SCCs) overexpress E2F1 (2, 43) and (vii) E2F inhibition makes SCC cell lines permissive to differentiation stimuli. Together, these data lend support to the hypothesis that E2F possesses biological properties expected of a modulator of squamous differentiation.

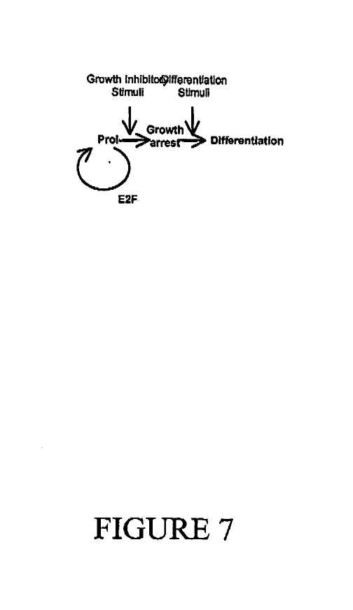

The data disclosed herein also show that the E2F pathway, especially the E2F polypeptide family, plays a dual role in promoting keratinocyte proliferation and modulating squamous differentiation. Not wishing to be bound by any one particular theory or mode of operation, it is proposed that components of the E2F pathway, especially members of the E2F polypeptide family, participate in proliferation control of undifferentiated keratinocytes and actively prevent them from undergoing differentiation. However, once a cell receives a signal to withdraw from the cell cycle and commits to differentiation (associated with loss of proliferation-competence), the components of the E2F pathway, especially the E2Fs, assume an active role as negative modulators of differentiation. The E2F pathway, in this model, is predicted to regulate the extent of the differentiation response and in concert with other differentiation-specific activators/repressors, the level of differentiation. In this way, the function of the E2F pathway, especially the E2Fs, may be considered analogous to the inhibitory function of the cyclin-dependent kinase inhibitor, p21Cipl/WAF1, in regulating primary mouse keratinocyte differentiation (60). However, studies with p21 suggest that it is actively involved in repression of differentiation. By contrast, the inventor's data indicate that E2F inhibition is required for the initiation of differentiation but is not sufficient to initiate differentiation. This indicates that terminal differentiation is not merely a result of growth arrest, but that differentiation is initiated and modulated by mechanisms yet to be understood that lie downstream of growth arrest and require an independent stimulus (see FIG. 7).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

1. Jones, S. J., Dicker, A. J., Dahler, A. L. & Saunders, N. A. (1997) J Invest Dermatol 109, 187-93.
2. Dicker, A. J., Popa, C., Dahler, A. L., Serewko, M. M., Hilditch-Maguire, P. A., Frazer, I. H. & Saunders, N. A. (2000) Oncogene 19, 2887-94.
3. Pierce, A. M., Fisher, S. M., Conti, C. J. & Johnson, D. G. (1998) Oncogene 16, 1267-76.
4. Woodworth, C. D., Wang, H., Simpson, S., Alvarez-Salas, L. M. & Notario, V. (1993) Cell Growth Differ 4, 367-76.
5. Saunders, N. A. & Jetten, A. M. (1994) J Biol Chem 269, 2016-22.
6. Dahler, A. L., Jones, S. J., Dicker, A. J. & Saunders, N. A. (1998) J Cell Physiol 177, 474-82.
7. Lane, E. B., Bartek, J., Purkis, P. E. & Leigh, I. M. (1985) Ann N Y Acad Sci 455, 241-58.
8. Rieger, M. & Franke, W. W. (1988) J Mol Biol 204, 841-56.
9. Marvin, K. W., George, M. D., Fujimoto, W., Saunders, N. A., Bernacki, S. H. & Jetten, A. M. (1992) Proc Natl Acad Sci USA 89, 11026-30.
10. Eckert, R. L., Crish, J. F., Banks, E. B. & Welter, J. F. (1997) J Invest Dermatol 109, 501-9.
11. Fuchs, E. & Raghavan, S. (2002) Nat Rev Genet 3, 199-209.
12. Kovesdi, I., Reichel, R. & Nevins, J. R. (1986) Cell 45, 219-28.
13. Kaelin, W. G., Jr., Krek, W., Sellers, W. R., DeCaprio, J. A., Ajchenbaum, F., Fuchs, C. S., Chittenden, T., Li, Y., Farnham, P. J., Blanar, M. A. & et al. (1992) Cell 70, 351-64.
14. Shan, B., Zhu, X., Chen, P. L., Durfee, T., Yang, Y., Sharp, D. & Lee, W. H. (1992) Mol Cell Biol 12, 5620-31.
15. Lees, J. A., Saito, M., Vidal, M., Valentine, M., Look, T., Harlow, E., Dyson, N. & Helin, K. (1993) Mol Cell Biol 13, 7813-25.
16. Helin, K., Lees, J. A., Vidal, M., Dyson, N., Harlow, E. & Fattaey, A. (1992) Cell 70, 337-50.
17. Ivey-Hoyle, M., Conroy, R., Huber, H. E., Goodhart, P. J., Oliff, A. & Heimbrook, D. C. (1993) Mol Cell Biol 13, 7802-12.
18. Beijersbergen, R. L., Kerkhoven, R. M., Zhu, L., Carlee, L., Voorhoeve, P. M. & Bernards, R. (1994) Genes Dev 8, 2680-90.
19. Ginsberg, D., Vairo, G., Chittenden, T., Xiao, Z. X., Xu, G., Wydner, K. L., DeCaprio, J. A., Lawrence, J. B. & Livingston, D. M. (1994) Genes Dev 8, 2665-79.
20. Itoh, A., Levinson, S. F., Morita, T., Kourembanas, S., Brody, J. S. & Mitsialis, S. A. (1995) Cell Mol Biol Res 41, 147-54. 21.
21. Buck, V., Allen, K. E., Sorensen, T., Bybee, A., Hijmans, E. M., Voorhoeve, P. M., Bemards, R. & La Thangue, N. B. (1995) Oncogene 11, 31-8.
22. Hijmans, E. M., Voorhoeve, P. M., Beijersbergen, R. L., van 't Veer, L. J. & Bernards, R. (1995) Mol Cell Biol 15, 3082-9.
23. Gaubatz, S., Wood, J. G. & Livingston, D. M. (1998) Proc Natl Acad Sci USA 95, 9190-5.
24. Trimarchi, J. M., Fairchild, B., Verona, R., Moberg, K., Andon, N. & Lees, J. A. (1998) Proc Natl Acad Sci USA 95, 2850-5.
25. Cartwright, P., Muller, H., Wagener, C., Holm, K. & Helin, K. (1998) Oncogene 17, 611-23.
26. Girling, R., Partridge, J. F., Bandara, L. R., Burden, N., Totty, N. F., Hsuan, J. J. & La Thangue, N. B. (1993) Nature 365, 468.
27. Wu, C. L., Zukerberg, L. R., Ngwu, C., Harlow, E. & Lees, J. A. (1995) Mol Cell Biol 15, 2536-46.
28. TZhang, Y. & Chellappan, S. P. (1995) Oncogene 10, 2085-93.
29. Helin, K., Wu, C. L., Fattaey, A. R., Lees, J. A., Dynlacht, B. D., Ngwu, C. & Harlow, E. (1993) Genes Dev 7, 1850-61.
30. Saunders, N. A., Popa, C., Serewko, M. M., Jones, S. J., Dicker, A. J. & Dahler, A. L. (1999) Expert Opin Investig Drugs 8, 1611-1621.
31. Ferreira, R., Magnaghi-Jaulin, L., Robin, P., Harel-Bellan, A. & Trouche, D. (1998) Proc Natl Acad Sci U S A 95, 10493-8.
32. Cobrinik, D., Whyte, P., Peeper, D. S., Jacks, T. & Weinberg, R. A. (1993) Genes Dev 7, 2392-404.
33. Weintraub, S. J., Prater, C. A. & Dean, D. C. (1992) Nature 358, 259-61.
34. Bates, S., Phillips, A. C., Clark, P. A., Stott, F., Peters, G., Ludwig, R. L. & Vousden, K. H. (1998) Nature 395, 124-5.
35. Russell, J. L., Powers, J. T., Rounbehler, R. J., Rogers, P. M., Conti, C. J. & Johnson, D. G. (2002) Mol Cell Biol 22, 1360-8.
36. Wang, D., Russell, J. L. & Johnson, D. G. (2000) Mol Cell Biol 20, 3417-24.
37. Moroni, M. C., Hickman, E. S., Denchi, E. L., Caprara, G., Colli, E., Cecconi, F., Muller, H. & Helin, K. (2001) Nat Cell Biol 3, 552-8.
38. Wang, J., Helin, K., Jin, P. & Nadal-Ginard, B. (1 995) Cell Growth Differ 6, 1299-306.
39. Guy, C. T., Zhou, W., Kaufman, S. & Robinson, M. 0. (1996) Mol Cell Biol 16, 685-93.
40. Fajas, L., Landsberg, R. L., Huss-Garcia, Y., Sardet, C., Lees, J. A. & Auwerx, J. (2002) Dev Cell 3, 39-49.
41. Saunders, N. A., Smith, R. J. & Jetten, A. M. (1993) Biochem Biophys Res Commun 197, 46-54.
42. Saunders, N. A., Bernacki, S. H., Vollberg, T. M. & Jetten, A. M. (1993) Mol Endocrinol 7, 387-98.
43. Dicker, A. J., Serewko, M. M., Dahler, A. L., Khanna, K. K., Kaur, P., Li, A., Strutton, G. M. & Saunders, N. A. (2000) Exp Cell Res 258, 352-60.
44. Rieger, M. & Franke, W. W. (1988) J Mol Biol 204, 841-56.
45. Andersen, B., Schonemann, M. D., Flynn, S. E., Pearse, R. V., 2nd, Singh, H. & Rosenfeld, M. G. (1993) Science 260, 78-82.
46. Xiao, Z. X., Chen, J., Levine, A. J., Modjtahedi, N., Xing, J., Sellers, W. R. & Livingston, D. M. (1995) Nature 375, 694-8.
47. Johnson, D. G., Schwarz, J. K., Cress, W. D. & Nevins, J. R. (1993) Nature 365, 349-52.
48. Khanna, K. K., Beamish, H., Yan, J., Hobson, K., Williams, R., Dunn, I. & Lavin, M. F. (1995) Oncogene 11, 609-18.
49. Saunders, N. A., Dicker, A. J., Jones, S. J. & Dahler, A. L. (1998) Cancer Res 58, 1646-9.
50. Adams, J. C. & Watt, F. M. (1988) J Cell Biol 107, 1927-38.
51. Jetten, A. M., George, M. A., Pettit, G. R., Herald, C. L. & Rearick, J. I. (1989) J Invest Dermatol 93, 108-15.
52. Bates, S., Parry, D., Bonetta, L., Vousden, K., Dickson, C. & Peters, G. (1994) Oncogene 9, 1633-40.
53. DeGregori, J., Leone, G., Miron, A., Jakoi, L. & Nevins, J. R. (1997) Proc Natl Acad Sci U SA 94, 7245-50.
54. Kowalik, T. F., DeGregori, J., Leone, G., Jakoi, L. & Nevins, J. R. (1998) Cell Growth Differ 9, 113-8.
55. Ziebold, U., Reza, T., Caron, A. & Lees, J. A. (2001) Genes Dev 15, 386-91.
56. Pierce, A. M., Schneider-Broussard, R., Gimenez-Conti, I. B., Russell, J. L., Conti, C. J. & Johnson, D. G. (1999) Mol Cell Biol 19, 6408-14.
57. Johnson, D. G., Cress, W. D., Jakoi, L. & Nevins, J. R. (1994) Proc Natl Acad Sci U S A 91, 12823-7.
58. Qin, X. Q., Livingston, D. M., Kaelin, W. G., Jr. & Adams, P. D. (1994) Proc Natl Acad Sci USA 91, 10918-22.
59. Shan, B. & Lee, W. H. (1994) Mol Cell Biol 14, 8166-73.
60. Di Cunto, F., Topley, G., Calautti, E., Hsiao, J., Ong, L., Seth, P. K. & Dotto, G. P. (1998) Science 280, 1069-72.
61. Paramio, J. M., Segrelles, C., Casanova, M. L. & Jorcano, J. L. (2000) J Biol Chem 275, 41219-26.
62. Lehman, T. A., Modali, R., Boukamp, P., Stanek, J., Bennett, W. P., Welsh, J. A., Metcalf, R. A., Stampfer, M. R., Fusenig, N., Rogan, E. M. & et al. (1993) Carcinogenesis 14, 833-9.
63. Boukamp, P., Petrussevska, R. T., Breitkreutz, D., Homung, J., Markham, A. & Fusenig, N. E. (1988) J Cell Biol 106, 761-71.
64. Apostolova, M. D., Ivanova, I. A., Dagnino, C., D'Souza, S. J. & Dagnino, L. (2002) J Biol Chem 277, 34471-9.
65. Gaubatz, S., Lees, J. A., Lindeman, G. J. & Livingston, D. M. (2001) Mol Cell Biol 21, 1384-92.
66. Morris, E. J. & Dyson, N. J. (2001) Adv Cancer Res 82, 1-54.
67. Yamanishi, K., Inazawa, J., Liew, F. M., Nonomura, K., Ariyama, T., Yasuno, H., Abe, T., Doi, H., Hirano, J. & Fukushima, S. (1992) J Biol Chem 267, 17858-63.
68. Mann, M. J., Whittemore, A. D., Donaldson, M. C., Belkin, M., Conte, M. S., Polak, J. F., Orav, E. J., Ehsan, A., Dell'Acqua, G. & Dzau, V. J. (1999) Lancet 354, 1493-8.
69. Ogawa H., Ishiguro K., Gaubatz S., Livingston D. M., & Nakatani Y. (2002) Science. 296(5570), 1132-6.
70. Trimarchi J. M., Fairchild B, Wen J. & Lees J. A. (2001) Proc Natl Acad Sci USA. 98(4), 1519-24.
71. de Bruin, A., Maiti, B., Jakoi, L., Timmers. C. & Leone, G. (2003) J. Biol. Chem., in press.

1. A method for modulating the proliferation and/or differentiation of an epithelial cell, comprising modulating an E2F pathway in the epithelial cell.

2. A method according to claim 1, wherein the E2F pathway is modulated by modulating the level or functional activity of an expression product of a gene selected from an E2F gene or a gene belonging to the same regulatory pathway as the E2F gene.

3. A method according to claim 2, wherein the gene belonging to the same regulatory pathway as the E2F gene is selected from the group consisting of CycD, CycE, RepA, cdk2, cdk4, Rb, E2F1, cdk1, p107, thymidylate synthase, dihydrofolate reductase, c-myc, transglutaminase type 1, Sp1 and Sp3.

4. A method according to claim 2, wherein the expression product is an E2F transcript that comprises a nucleotide sequence corresponding to any one of E2F1, E2F2, E2F3, E2F4, E2F5, E2F6 and E2F7.

5. A method according to claim 2, wherein the expression product is an E2F polypeptide that comprises an amino acid sequence corresponding to any one of E2F1, E2F2, E2F3, E2F4, E2F5, E2F6 and E2F7.

6. A method according to claim 1, wherein the epithelial cell is a squamous epithelial cell.

7. A method according to claim 6, wherein the squamous epithelial cell is from the epidermis, oral mucosa, oesophageal, vaginal, tracheal or corneal epithelia.

8. A method according to claim 1, wherein the E2F pathway is antagonized using an antagonist of the pathway to thereby inhibit or arrest the proliferation of the cell and to potentiate or induce its differentiation.

9. A method according to claim 1, wherein the E2F pathway is antagonized using an antagonist of the pathway, which is selected from the group consisting of an antisense RNA molecule, an antisense DNA molecule, a ribozyme, an RNAi molecule, a dominant negative polypeptide and antigen-binding molecule.

10. A method according to claim 1, wherein the E2F pathway is antagonized using an antagonist of the pathway, which reduces or abrogates the level or functional activity of an expression product of an E2F activator gene or of a gene that is directly or indirectly modulated by an expression product of the E2F activator gene.

11. A method according to claim 10, wherein the antagonist selected from the group consisting of an anti-E2F1 antisense RNA molecule, an anti-E2F2 antisense RNA molecule, an anti-E2F3 antisense RNA molecule, an anti-E2F1 antisense DNA molecule, an anti-E2F2 antisense DNA molecule, an anti-E2F3 antisense DNA molecule, an anti-E2F1 ribozyme, an anti-E2F2 ribozyme, an anti-E2F3 ribozyme, an anti-E2F1 RNAi molecule, an anti-E2F2 RNAi molecule, an anti-E2F3 RNAi molecule, an anti-E2F1 dominant negative polypeptide, an anti-E2F2 dominant negative polypeptide, an anti-E2F3 dominant negative polypeptide, an anti-E2F1 antigen-binding molecule, an anti-E2F2 antigen-binding molecule, an anti-E2F3 antigen-binding molecule, an E2F6 polypeptide, E2F7 polypeptide and a construct from which any one of these is expressible.

12. A method according to claim 1, wherein the E2F pathway is antagonized using an antagonist of the pathway, which modulates the level or functional activity of an expression product of a gene belonging to the E2F pathway by at least 10% relative to the level or functional activity in the absence of the antagonist.

13. A method according to claim 1, wherein the E2F pathway is antagonized using an antagonist of the pathway, which enhances the level or functional activity of an expression product of an E2F repressor gene selected from the group consisting of E2F4, E2FS, E2F6 and E2F7.

14. A method according to claim 13, wherein the antagonist is selected from the group consisting of a polynucleotides from which the E2F repressor gene is expressible and an expression product of the E2F repressor gene.

15. A method according to claim 1, wherein the epithelial cell is a cancer cell.

16. A method according to claim 1, wherein the epithelial cell is a skin cancer cell.

17. A method according to claim 1, wherein the epithelial cell is a squamous cell carcinoma cell.

18. A method according to claim 2, wherein the antagonist antagonizes the E2F pathway as determined by: contacting a preparation comprising at least a portion of an expression product of the gene, or a genetic sequence that modulates the expression of the gene, with the antagonist; and detecting a change in the level or functional activity of the at least a portion of the expression product, or of a product expressed from the genetic sequence.

19. A method according to claim 2, wherein the antagonist antagonizes the E2F pathway as determined by: contacting a preparation comprising an E2F polypeptide or a biologically active fragment thereof, or a variant or derivative of these, or a genetic sequence that modulates the expression of an E2F gene; and detecting a decrease in the level or functional activity of the E2F polypeptide or biologically active fragment thereof, or variant or derivative, or of a product expressed from the genetic sequence.

20. A method according to claim 1, further comprising exposing the epithelial cell to a differentiation stimulus that stimulates or otherwise induces the differentiation of the epithelial cell.

21. A method according to claim 20, wherein the differentiation stimulus is selected from high calcium concentrations, phorbol esters, butyric acid, activators of PPRγ-type receptors, macrocyclic diterpenes selected from compounds of the ingenane, pepluane and jatrophane families, indirubins, histone deacetylase inhibitors, retinoids and TGFβ1.

22. A method according to claim 20, wherein the differentiation stimulus is 12-O-tetradecanoylphorbol-13 -acetate.

23. A method according to claim 20, wherein the differentiation stimulus is an activator of a PPRγ-type receptor, which is selected from the group consisting of 5-{4-[2-(methyl-pyrid-2-ylamino)ethoxy]benzl}thiazolidine-2,4-dione, 3-{4-[2-(benzoxazol-2-ylmethylamino)ethoxy]phenyl}-2-ethoxypropionic acid, (+)-3 -{4-[2-(benzoxazol-2-ylmethylamino)ethoxy]phenyl}-2-ethoxypropionic acid, and (−)-3-{4-[2-(benzoxazol-2-ylmethylamino)ethoxy]phenyl}-2-ethoxypropionic acid.

24. A method according to claim 20, wherein the differentiation stimulus is meisoindigo.

25. A composition for modulating the proliferation and/or differentiation of an epithelial cell, comprising an agent that modulates an E2F pathway and a differentiation-stimulating agent that stimulates or otherwise induces the differentiation of an epithelial cell.

26. A composition according to claim 25, further comprising a pharmaceutically acceptable carrier.

27. A conjugate comprising an agent that modulates the activity of an E2F pathway component and an agent that is immuno-interactive with a surface protein whose expression is upregulated on a skin cancer cell.

28. A conjugate according to claim 27, wherein the agent is an antagonist of the E2F pathway.

29. A conjugate according to claim 27, wherein the skin cancer cell is a squamous cell carcinoma.

30. A conjugate according to claim 29, wherein the surface protein is an EGF receptor.

31. A method for treating or preventing a skin cancer or tumor of epithelial origin in a patient, comprising administering to the patient an effective amount of an agent that antagonizes the function of an E2F pathway.

32. A method according to claim 31, further comprising separately, sequentially or simultaneously administering a differentiation stimulus that stimulates or otherwise induces the differentiation of an epithelial cell.

33. A method according to claim 31, wherein the skin cancer or tumor of epithelial origin is a squamous cell carcinoma.

34. A method for identifying an agent that modulates the proliferation and/or differentiation of an epithelial cell, comprising:
contacting a preparation with a test agent, wherein the preparation comprises (i) a polypeptide comprising an amino acid sequence corresponding to at least a biologically active fragment of a polypeptide component of the E2F pathway, or to a variant or derivative thereof; or (ii) a polynucleotide comprising at least a portion of a genetic sequence that regulates the component, which is operably linked to a reporter gene; and
detecting a change in the level or functional activity of the polypeptide component, or an expression product of the reporter gene, relative to a normal or reference level or functional activity in the absence of the test agent, which indicates that the agent modulates the proliferation and/or differentiation of an epithelial cell.

35. A method for identifying an agent that modulates the proliferation and/or differentiation of an epithelial cell, comprising:
contacting a first sample of cells expressing an E2F pathway component and measuring at least one marker;
contacting a second sample of cells expressing the component with an agent and measuring the marker(s); and
comparing the marker(s) of the first sample of cells with the marker(s) of the second sample of cells, wherein the marker(s) is/are selected from components of the E2F pathway and optionally from markers associated with the proliferation and/or differentiation of the epithelial cell.

36. A method according to claim 34 or claim 35, further comprising administering the agent to an animal model, or a to human, and measuring the animal's responsiveness to the agent.

37. A method according to claim 36, wherein the responsiveness is measured by measuring the proliferation and/or differentiation of epithelial cells in the patient.

38. A method for inhibiting the proliferation and/or differentiation of an epithelial cancer cell, comprising antagonising an E2F pathway in the cancer cell.

39. A method for inhibiting the proliferation and/or differentiation of an epithelial skin cancer cell, comprising antagonising an E2F pathway in the skin cancer cell.

40. A method for inhibiting the proliferation and/or differentiation of a squamous cell carcinoma cell, comprising antagonising an E2F pathway in the carcinoma cell.

41. A method for inhibiting the proliferation and/or differentiation of an epithelial cancer cell, comprising antagonising an E2F pathway in the cancer cell with an antagonist of the pathway, which reduces or abrogates the level or functional activity of an expression product of an E2F activator gene or of a gene that is directly or indirectly modulated by an expression product of the E2F activator gene.

42. A method according to claim 41, wherein the antagonist is selected from the group consisting of an anti-E2F1 antisense RNA molecule, an anti-E2F2 antisense RNA molecule, an anti-E2F3 antisense RNA molecule, an anti-E2F1 antisense DNA molecule, an anti-E2F2 antisense DNA molecule, an anti-E2F3 antisense DNA molecule, an anti-E2F1 ribozyme, an anti-E2F2 ribozyme, an anti-E2F3 ribozyme, an anti-E2F1 RNAi molecule, an anti-E2F2 RNAi molecule, an anti-E2F3 RNAi molecule, an anti-E2F1 dominant negative polypeptide, an anti-E2F2 dominant negative polypeptide, an anti-E2F3 dominant negative polypeptide, an anti-E2F1 antigen-binding molecule, an anti-E2F2 antigen-binding molecule, an anti-E2F3 antigen-binding molecule, an E2F6 polypeptide, E2F7 polypeptide and a construct from which any one of these is expressible.

43. A method for inhibiting the proliferation and/or differentiation of an epithelial cancer cell, comprising antagonising an E2F pathway in the cancer cell with an antagonist of the pathway, which enhances the level or functional activity of an expression product of an E2F repressor gene selected from the group consisting of E2F4, E2F5, E2F6 and E2F7.

44. A method according to claim 43, wherein the antagonist is selected from the group consisting of a polynucleotides from which the E2F repressor gene is expressible and an expression product of the E2F repressor gene.

45. A method for diagnosing the presence, or risk of development, of a skin cancer or tumor of epithelial origin in a test subject, comprising detecting in the test subject aberrant expression of at least one gene selected from an E2F gene or a gene belonging to the same regulatory pathway as the E2F gene.

46. A method according to claim 45, wherein the gene is selected from the group consisting of E2F1, E2F2 and E2F3.

47. A method according to claim 45, wherein skin cancer or tumor of epithelial origin is a squamous cell carcinoma.

48. A method according to claim 45, wherein the aberrant expression is detected by: (1) measuring in a biological sample obtained from the test subject the level or functional activity of an expression product of at least one E2F pathway marker gene and (2) comparing the measured level or functional activity of each expression product to the level or functional activity of a corresponding expression product in a reference sample obtained from one or more normal subjects or from one or more subjects lacking the skin cancer or tumor of epithelial origin, wherein a difference in the level or functional activity of the expression product in the biological sample as compared to the level or functional activity of the corresponding expression product in the reference sample is indicative of the presence, or risk of development, of the skin cancer or tumor of epithelial origin in the test subject.

49. A method according to claim 45, wherein the aberrant expression is detected by detecting increased expression of an E2F pathway marker gene selected from the group consisting of E2F1, E2F2 and E2F3.

50. A method according to claim 49, wherein the aberrant expression is detected by detecting increased expression of E2F1.

51. A method according to claim 48, wherein the aberrant expression is detected when the measured level or functional activity of the or each expression product is at least 10% higher or lower than the measured level or functional activity of the or each corresponding expression product.
